(19) United States
(12) Patent Application Publication
(10) Pub. No.: US 2020/0054684 A1
(43) Pub. Date: Feb. 20, 2020

(54) NON-MESENCHYMAL HUMAN LUNG STEM CELLS AND METHODS OF THEIR USE FOR TREATING RESPIRATORY DISEASES

(71) Applicant: AAL SCIENTIFICS, INC., New York, NY (US)

(72) Inventors: Piero ANVERSA, New York, NY (US); Annarosa LERI, New York, NY (US)

(73) Assignee: AAL SCIENTIFICS, INC., New York, NY (US)

(21) Appl. No.: 16/346,932

(22) PCT Filed: Nov. 2, 2017
(86) PCT No.: PCT/US2017/059684

§ 371 Date: May 2, 2019

Related U.S. Application Data
(60) Provisional application No. 62/416,562, filed on Nov. 2, 2016.

(51) Int. Cl. A61K 35/42; C12N 5/071; A61P 11/00

(52) U.S. Cl. CPC A61K 35/42; C12N 2501/599; A61P 11/00; C12N 5/0689

(57) ABSTRACT

Embodiments of the invention relate to human, non-mesenchymal c-kit positive lung stem cells negative for the CD44, CD73 and CD105 markers of the mesenchymal stromal cell lineage (non-mhLSCs) and their therapeutic use in the treatment and/or prevention of lung diseases or disorders. Provided herein are compositions comprising non-mhLSCs and methods of preparing and using non-mhLSCs for the treatment and/or prevention of lung diseases or disorders.

This application is a National Stage of International Patent Application No. PCT/US2017/059684, filed Nov. 2, 2017, which claims priority to and benefit of U.S. Provisional Patent Application No. 62/416,562, filed on Nov. 2, 2016. The contents of both applications are herein incorporated by reference in their entirety.

### BACKGROUND OF INVENTION

Every year over 400,000 Americans die from some type of lung disease and that number is larger worldwide. Moreover, death rates due to lung diseases are currently increasing. According to the American Lung Association, chronic obstructive pulmonary disease (COPD) is expected to become the third leading cause of death by 2020.

A lung disease is any disease or disorder where lung function is impaired. Lung diseases can be caused by long-term and/or immediate exposure to, among other things, smoking, secondhand smoke, air pollution, occupational hazards such as asbestos and silica dust, carcinogens that trigger tumor growth, infectious agents, and over reactive immune defenses. Over a period of time, lung tissues including the airway and blood vessels become damaged such that there is not enough healthy tissue to support adequate gaseous exchange to supply sufficient oxygen for all the cells in the body for basic function. In essence, these people “suffocate” slowly to death. Therefore, lung disease can be a life-threatening illness or condition.

There are many types of lung diseases including: (A) Obstructive lung diseases such as asthma and COPD which includes chronic bronchitis and emphysema. These all affect a person's airways and limit or block the flow of air in or out of the lungs; (B) Infectious illnesses such as pneumonia, influenza, respiratory syncytial virus (RSV) and tuberculosis (TB). Bacteria or viruses cause these diseases that can also affect the membrane (or pleura) that surround the lungs; (C) Lung cancer which is a disease characterized by uncontrolled growth and spread of abnormal cells; (D) Respiratory failure, pulmonary edema, pulmonary embolism and pulmonary hypertension. These conditions are caused by problems with the normal gas exchange and blood flow in the lungs; and (E) Pulmonary fibrosis and sarcoidosis. These are diseases characterized by stiffening and scarring of the lungs and occupational diseases, such as mesothelioma and asbestosis, caused by expo-sure to hazardous substances.

Currently, all treatments for lung diseases are mainly palliative, where the emphasis is on maintaining quality of life through symptom management. Lung transplantation is the therapeutic measure of last resort for patients with end-stage lung disease who have exhausted all other available treatments without improvement. As of 2005, the most common reasons for lung transplantation in the United States were: 27% chronic obstructive pulmonary disease (COPD), including emphysema; 16% idiopathic pulmonary fibrosis; 14% cystic fibrosis; 12% idiopathic (formerly known as “primary”) pulmonary hypertension; 5% alpha 1-antitrypsin deficiency; 2% replacing previously transplanted lungs that have since failed; and 24% other causes, including bronchiectasis and sarcoidosis.

Lung transplantation or pulmonary transplantation is a surgical procedure in which a patient's diseased lungs are partially or totally replaced by lungs which come from a donor. While lung transplants carry certain associated risks, they can also extend life expectancy and enhance the quality of life for end-stage pulmonary′ patients. Often, a combined heart and lung transplantation is done because both organs are intricately connected physically and functionally, and a dual transplant greatly increases the success of the transplant. However, the availability of a dual or even a single organ for transplant is very rare because certain criteria for potential donors must be fulfilled, e.g. health of donor, size match, the donated lung or lungs must be large enough to adequately oxygenate the patient, but small enough to fit within the recipient's chest cavity, age, and blood type. As a result, patients often die while on the waiting list.

Even for those lucky enough to receive a transplant, the average survival of a lung transplant patient is about 5 to 10 years which is relatively low compared to other type of organ transplantation; for lung transplant 53.4% and 28.4% respectively, and for heart-lung transplant 46.5% and 28.3% respectively (data taken from 2008 OPTN/SRTR Annual Report, US Scientific Registry of Transplant Recipients).

Sometimes, a lung transplant is not an option. Not all patients with lung disease make good candidates for lung transplant. Sometimes, despite the severity of a patient's respiratory′ condition, certain pre-existing conditions may make a person a poor candidate for lung transplantation. These conditions include: concurrent chronic illness (e.g. congestive heart failure, kidney disease, liver disease); current infections, including HIV and hepatitis, current or recent cancer, current use of alcohol, tobacco, or illegal drugs; age; within an acceptable weight range (marked undernourishment or obesity are both associated with increased mortality); psychiatric conditions; history of noncompliance with medical instructions; and previous multiple failed lung transplantation.

In addition for those patients having under gone a lung transplant, there may be other complications associated with the transplant which include organ rejection, post-transplant lymphoproliferative disorder, a form of lymphoma due to the immune suppressants, and gastrointestinal inflammation and ulceration of the stomach and esophagus.

Other solutions that supplement the palliative care that keep these patients alive are desirable, for example, for those on the waiting list, and especially those patients that do not qualify for lung transplant.

Human stern cells and methods of preparing and using them are disclosed in WO 2012/047951, which is herein incorporated in its entirety for all purposes. Additional solutions that keep the patients off the lung transplant waiting list are also desired.

### SUMMARY OF THE INVENTION

Embodiments of the invention relate to human stem cells and methods of preparing and using them.

Embodiments of the present invention are based on the discovery of a pool of c-kit-positive human lung stem cells (hLSCs) that is composed of one cell class, non-mesenchymal hLSCs (non-mhLSCs), that is negative for the mesenchymal epitopes CD44, CD73 and CD105, and another cell class, mesenchymal-like hLSCs (ml-hLSCs), that expresses these epitopes and differentiates into adipocytes, chondrocytes, osteocytes and fibroblasts. Both cell types possess the properties of tissue specific adult stem cells, i.e., self-renewal and clonogenicity.

Embodiments of the present invention provide solutions to the problem of donor lung shortages and the problem of ineligibility for a lung transplant of a subject having a lung disease or is at risk of developing a lung disease in the future. Specifically, the problems are solved by implanting non-mhLSCs to defective and/or damaged lungs in order to promote lung repair and regeneration and to extend the life of the subject till a donor lung becomes available in the first case or for as long as possible with acceptable quality of life in the second case.

Accordingly, in one aspect, the invention provides a pharmaceutical composition comprising: an enriched population of isolated c-kit positive lung stem cells from a human lung tissue sample wherein the c-kit positive lung stem cells are negative for the CD44, CD73 and CD105 markers of the mesenchymal stromal cell lineage (non-mhLSCs); and a pharmaceutically acceptable carrier. In one embodiment, the pharmaceutical composition is formulated for intrapulmonary administration, systemic administration, intravenous administration, or a combination thereof. In another embodiment, the intrapulmonary administration is intratracheal or intranasal administration. in a further embodiment, the population of non-mhLSCs is further expanded ex vivo.

In another aspect, the invention provides a method of preparing an isolated population of lung stem cells positive for c-kit and negative for the CD44, CD73 and CD105 markers of the mesenchymal stromal cell lineage (non-mhLSCs), wherein the non-mhLSCs are in a pool of c-kit-positive human lung stem cells (hLSCs) comprised of non-mhLSCs and mesenchymal-like lung stem cells that are positive for c-kit and the CD44, CD73 and CD105 markers (ml-hLSCs), the method comprising: obtaining human lung tissue from a subject; selecting non-mhLSCs from the pool of hLSCs from the human lung tissue; and proliferating said cells in a culture medium.

In another aspect, the invention provides a method of proliferating an isolated population of lung stem cells positive for c-kit and negative for the CD44, CD73 and CD105 markers of the mesenchymal stromal cell lineage (non-mhLSCs), wherein the non-mhLSCs are in a pool of c-kit-positive human lung stem cells (hLSCs) comprised of non-mhLSCs and mesenchymal-like lung stem cells that are positive for c-kit and the CD44, CD73 and CD105 markers (ml-hLSCs), the method comprising: selecting at least one non-mhLSC from the pool of hLSCs from a human lung tissue sample; introducing said at least one selected non-mhLSC to a culture medium; and proliferating said at least one selected non-mhLSC in the culture medium.

In another aspect, the invention provides a method of repairing and/or regenerating damaged lung tissue in a subject in need thereof comprising: extracting a population of stem cells positive for c-kit and negative for the CD44, CD73 and CD105 markers of the mesenchymal stromal cell lineage (non-mhLSCs) from lung tissue; culturing and expanding said population of non-mhLSCs; and administering a dose of said extracted and expanded population of non-mhLSCs to an area of damaged lung tissue in the subject effective to repair and/or regenerate the damaged lung tissue.

In another aspect, the invention provides a method for treating or preventing a lung disease or disorder in a subject in need thereof, comprising: obtaining a human lung tissue from the subject in need thereof or from a different subject; extracting a population of stem cells positive for c-kit and negative for the CD44, CD73 and CD105 markers of the mesenchymal stromal cell lineage (non-mhLSCs) from said lung tissue; expanding said population of non-mhLSCs; and administering said expanded population of non-mhLSCs to the subject in need thereof.

In another aspect, the invention provides a composition for use in treating and/or preventing a lung disease or disorder in a subject, the composition comprising an enriched population of isolated c-kit positive lung stem cells from a human lung tissue sample wherein the c-kit positive lung stem cells are negative for the CD44, CD73 and CD105 markers of the mesenchymal stromal cell lineage (non-mhLSCs). In one embodiment, the enriched population of isolated non-mhLSCs also comprises lung progenitor cells and lung precursor cells. In one embodiment, the composition is formulated for intrapulmonary administration, systemic administration, intravenous administration, or a combination thereof. In another embodiment, the intrapulmonary administration is intratracheal or intranasal administration. In a further embodiment, the enriched population of isolated non-mhLSCs is further expanded ex vivo.

In one embodiment of all aspects of the treatment or prevention methods, the population of non-mhLSCs is derived from the subject in need of treatment or prevention. In one embodiment, the population of non-mhLSCs is autologous.

In one embodiment of all aspects of the treatment or prevention methods, the population of non-mhLSCs is derived from one subject and administered to another subject, meaning that the donor of the non-mhLSCs is not the same person as the recipient of the non-mhLSCs. It is understood that the donor and recipient should be antigen matched for such transplant, and the matching criteria and methods are well known in the art. The donor non-mhLSCs ideally should be allogeneic and HLA type matched to a recipient.

Accordingly, in one embodiment, the invention provides a method for treating or preventing a lung disease or disorder in a subject in need thereof, the method comprising obtaining a lung tissue sample from a first subject; extracting a population of stem cells positive for c-kit and negative for the CD44, CD73 and CD105 markers of the mesenchymal stromal cell lineage (non-mhLSCs) from the lung tissue sample; expanding the population of non-mhLSCs; and administering the population of non-mhLSCs to a second subject for the non-mhLSCs to take up residence in the lungs and repairs/reconstitutes/and/or generates pulmonary cells and tissues in the lung of the second subject. In one embodiment of this treatment method, the second subject is at least one HLA type matched with the first subject, the donor of the non-mhLSCs.

In one embodiment of all aspects of the treatment or prevention methods described, the administered population of lung stem cells positive for c-kit and negative for the CD44, CD73 and CD105 markers of the mesenchymal stromal cell lineage (non-mhLSCs) repairs, reconstitutes or generates pulmonary epithelium, pulmonary vasculature/pulmonary endothelium and pulmonary alveoli in the lung of the subject.

In another embodiment of all aspects of the treatment or prevention methods described, the administered population of lung stem cells positive for c-kit and negative for the CD44, CD73 and CD105 markers of the mesenchymal stromal cell lineage (non-mhLSCs) restores the structural and functional integrity of the lung of the subject.

In one embodiment of all aspects of the compositions and methods described, the lung tissue is from a human. In another embodiment of all aspects of the compositions and methods described, the human lung tissue is an adult lung tissue.

In one embodiment of all aspects of the compositions and methods described, the lung tissue sample is cryopreserved prior to the selection of non-mhLSCs. Cryopreservation can also be performed on the isolated non-mhLSCs from the lung tissue sample prior to the expansion in culture medium and on the expanded non-mhLSCs.

In one embodiment of all aspects of the compositions and methods described, the selection of non-mhLSCs is performed using an antibody against c-kit. In another embodiment of all aspects of the compositions and methods described, the selection of non-mhLSCs further comprises negative selection for the CD44, CD73 and CD105 markers of the mesenchymal stromal cell lineage.

In one embodiment of all aspects of the compositions and methods described, the population of non-mhLSCs can differentiate into alveolar epithelial cells, capillary endothelial cells, or a combination thereof. In a further embodiment, the population of non-mhLSCs is self-renewing and clonogenic.

In one embodiment of all aspects of the compositions and methods described, the selection of c-kit positive cell is by flow cytometry.

In another embodiment of all aspects of the compositions and methods described, the selection of non-mhLSCs is by immunomagnetic selection with c-kit antibodies conjugated to beads.

In one embodiment of all aspects of the compositions and methods described herein, the population of lung stem cells positive for c-kit and negative for the CD44, CD73 and CD105 markers of the mesenchymal stromal cell lineage can be cryopreserved.

In one embodiment of all aspects of the compositions and methods described herein, the lung disease or disorder is one or more of chronic obstructive pulmonary disease (COPD), idiopathic pulmonary fibrosis (IPF), or progressive pulmonary fibrosis (PPF).

In one embodiment of all aspects of the treatment or prevention methods described, the therapeutic method further comprises administering at least one therapeutic agent, e.g., one that decreases pulmonary hypertension.

In one embodiment of all aspects of the treatment or prevention methods described, the therapeutic method further comprises selecting a subject who is suffering from a lung disorder prior to administering the population enriched for non-mhLSCs.

In one embodiment of all aspects of the treatment or prevention methods described, the therapeutic method further comprises selecting a subject in need of restoring the structural and functional integrity of a damaged lung prior to administering the non-mhLSCs.

In one embodiment of all aspects of the treatment or prevention methods described, the therapeutic method further comprises selecting a subject in need of treatment, prevention or repair or reconstitution or generation of pulmonary vasculature or pulmonary epithelium, pulmonary endothelium, or pulmonary alveoli prior to administering the non-mhLSCs. Subjects such as those who smoke and/or have been exposed to asbestos are at high risk for developing various lung diseases and they would be candidates for the method to prevent their lung diseases from developing and also to prevent the disease from progressing once the disease has started.

In one embodiment of all aspects of the therapeutic methods described herein, the administration is intrapulmonary administration, systemic administration, intravenous administration, or a combination thereof.

In one embodiment of all aspects of the therapeutic methods described herein, the intrapulmonary administration is either intratracheal or intranasal administration.

### BRIEF DESCRIPTION OF THE DRAWINGS

FIG. 1 shows lung samples. Specimens of control lung (upper left) and IPF/PPF lung (lower left) were enzymatically digested to obtain a single cell suspension. The mid-density cells contain human lung stem cells.

FIG. 2A shows human lung stem cell (hLSC) classes. Dot plots illustrate that the compartment of hLSCs contains a population of non-mesenchymal c-kit-positive cells which do not express the epitopes CD44/CD73/CD105 (non-mhLSCs) and a category of c-kit-positive cells that expresses CD44/CD73/CD105, i.e., mesenchymal-like hLSCs (ml-hLSCs). These two cell populations are present in control. IPF/PPF and COPD lungs.

FIG. 2B shows non-mhLSC and ml-hLSC clones. Clones from control and IPF/PPF non-mhLSCs display typical features of stem cell-formed colonies. They have a compact round profile and only occasionally an irregular shape, as shown in the third and fourth clones on the right. However, ml-hLSCs generate only non-circular irregularly shaped clones with refractive edges.

FIG. 2C shows immunohistochemistry of non-mhLSC and ml-hLSC clones. Circular clones are composed of undifferentiated cells intensely positive for c-kit, high nucleus-to-cytoplasm ratio and negative for CD44/CD73/CD105 (left panel). The non-circular clones are characterized by cells weakly labeled for c-kit, low nucleus-to-cytoplasm ratio and positive for CD44/CD73/CD105 (center and right panels).

FIG. 2D shows the proportion of non-mhLSCs and ml-hLSCs in control, IPF/PPF and COPD lungs; data are mean±SD.

FIG. 3 shows an invasion assay. Using a matrigel-coated transwell chamber (see scheme at top of FIG. 3), a cell invasion assay was performed to determine the invasive capabilities of differentiating control and IPF/PPF non-mhLSCs and ml-hLSCs exposed to fetal bovine serum (FBS). IPF/PPF ml-hLSCs invaded the basement membrane matrigel at a very high rate. The migrated ml-hLSCs acquired the myofibroblast phenotype and were positive for both α-smooth muscle actin (α-SMA; right panel, red) and procollagen (not shown).

### DETAILED DESCRIPTION OF THE INVENTION

Embodiments of the present invention are based on the discovery of a pool of c-kit-positive human lung stem cells (hLSCs) that is composed of one cell class, non-mesenchymal hLSCs (non-mhLSCs), that is negative for the mesenchymal epitopes CD44, CD73 and CD105, and another cell class, mesenchymal-like hLSCs (ml-hLSCs), that expresses epitopes CD44, CD73 and CD105. Both cell types possess the properties of tissue specific adult stem cells, i.e., self-renewal and clonogenicity.

Of relevance, clonal non-mhLSCs differentiate into alveolar epithelial cells and capillary endothelial cells, while clonal ml-hLSCs do not acquire the epithelial and vascular cell lineages. Clonal ml-hLSCs instead differentiate into adipocytes, chondrocytes, osteocytes and fibroblasts. Importantly, a subset of functional non-mhLSCs is present in the diseased lung, and these cells can be harvested and propagated in vitro. In one embodiment, autologous cell therapy using non-mhLSCs can be carried out to reverse the devastating consequences of lung diseases such as idiopathic pulmonary fibrosis/progressive pulmonary fibrosis (IPF/PPF) and chronic obstructive pulmonary disease (COPD).

As it is well known, stem cells, by virtue of its properties, give rise to all the cells and tissues of the body. Therefore, stem cells can be used to repair or speed up the repair of a damaged and/or defective lung. If sufficient amount of adult lung stem cells (LSCs) can be obtained, this amount of adult (LSCs) can be used to repair damaged and/or defective lungs by building new tissues in the lungs. In a defective and/or damaged lung, there may be few or absent LSCs. Since adult LSCs self-renew, the implanted adult LSCs will colonize and populate niches in the defective and/or damaged lung. By being clonal, self-renewing and able to differentiate into alveolar epithelial cells, capillary endothelial cells, or a combination thereof, the implanted non-mhLSCs will also divide and differentiate to produce all new lung cells and tissues. Therefore, a population of isolated non-mhLSCs or a composition comprising a population of isolated non-mhLSCs can be used for treatment or prevention of a lung disease in a subject.

Accordingly, the problem of a subject with a lung disease dying prematurely before a donor lung becomes available or because of ineligibility for a lung transplant is solved by implanting non-mhLSCs to the defective and/or damaged lungs of the subject in order to promote de novo lung repair and regeneration. The de novo lung repair and regeneration can extend the life of the subject until a donor lung becomes available in the first case or sustain life of the subject for as long as possible with an acceptable quality of life in the second case.

Accordingly, in one embodiment, the invention provides an enriched population of isolated c-kit positive lung stem cells, called non-mhLSCs, from a human lung tissue sample wherein the c-kit positive lung stem cells are negative for the CD44, CD73 and CD105 markers of the mesenchymal stromal cell lineage. In another embodiment, the population of isolated cells is substantially enriched for c-kit positive lung cells, which comprises predominantly (≥70%) of LSCs.

In one embodiment, the population of isolated cells that is substantially enriched for non-mhLSCs also comprises a very small number of lung progenitor cells and lung precursor cells.

In one embodiment, provided herein is a pharmaceutical composition comprising: an enriched population of isolated c-kit positive lung stem cells from a human lung tissue sample wherein the c-kit positive lung stem cells are negative for the CD44, CD73 and CD105 markers of the mesenchymal stromal cell lineage (non-mhLSCs); and a pharmaceutically acceptable carrier. In one embodiment, the pharmaceutical composition is formulated for intrapulmonary administration, systemic administration, intravenous administration, or a combination thereof. In another embodiment, the intrapulmonary administration is intratracheal or intranasal administration. In a further embodiment, the population of non-mhLSCs is further expanded ex vivo.

In one embodiment, provided herein is a composition for use in treating and/or preventing a lung disease or disorder in a subject, the composition comprising an enriched population of isolated c-kit positive lung stem cells from a human lung tissue sample wherein the c-kit positive lung stem cells are negative for the CD44, CD73 and CD105 markers of the mesenchymal stromal cell lineage (non-mhLSCs). In one embodiment, the enriched population of isolated non-mhLSCs also comprises lung progenitor cells and lung precursor cells. In one embodiment, the composition is formulated for intrapulmonary administration, systemic administration, intravenous administration, or a combination thereof. In another embodiment, the intrapulmonary administration is intratracheal or intranasal administration. In a further embodiment, the enriched population of isolated non-mhLSCs is further expanded ex vivo. In another embodiment of this composition, the composition further comprises a pharmaceutically acceptable carrier.

In one embodiment, the invention provides a method of preparing an isolated population of lung stem cells positive for c-kit and negative for the CD44, CD73 and CD105 markers of the mesenchymal stromal cell lineage (non-mhLSCs), wherein the non-mhLSCs are in a pool of c-kit-positive human lung stem cells (hLSCs) comprised of non-mhLSCs and mesenchymal-like lung stem cells that are positive for c-kit and the CD44, CD73 and CD105 markers (ml-hLSCs), the method comprising: obtaining human lung tissue from a subject; selecting non-mhLSCs from the pool of hLSCs from the human lung tissue; and proliferating said cells in a culture medium. In another embodiment, the number of non-mhLSCs increases by at least two fold over the initial amount selected, preferably by more than two fold.

In one embodiment, the invention provides a method of obtaining an enriched population of isolated c-kit positive lung stem cells from a human lung tissue sample wherein the c-kit positive lung stem cells are negative for the CD44, CD73 and CD105 markers of the mesenchymal stromal cell lineage (non-mhLSCs), the method comprising cryopreserving a specimen of lung tissue obtained from a subject; thawing the cryopreserved specimen at a later date; selecting at least one c-kit positive non-mhLSC from the specimen of lung tissue; and proliferating the selected non-mhLSCs in a culture medium whereby the number of non-mhLSCs at least doubles over the initial amount selected, preferably by more than double.

In one embodiment, the invention provides a method of proliferating an isolated population of lung stem cells positive for c-kit and negative for the CD44, CD73 and CD105 markers of the mesenchymal stromal cell lineage (non-mhLSCs), wherein the non-mhLSCs are in a pool of c-kit-positive human lung stem cells (hLSCs) comprised of non-mhLSCs and mesenchymal-like lung stem cells that are positive for c-kit and the CD44, CD73 and CD105 markers (ml-hLSCs), the method comprising: selecting at least one non-mhLSC from the pool of hLSCs from a human lung tissue sample; introducing said at least one selected non-mhLSC to a culture medium; and proliferating said at least one selected non-mhLSC in the culture medium. In one embodiment, the number of non-mhLSCs increases by at least two fold over the initial amount selected, preferably by more than two fold.

In another embodiment, the invention provides methods of use of this population of isolated non-mhLSCs from lung tissue or use of a pharmaceutical composition comprising an enriched population of isolated non-mhLSCs from lung tissue. For example, the population of isolated non-mhLSCs can be used for the repair, regeneration and/or treatment of lung diseases and disorders.

The inventors of the disclosure have found that the non-mesenchymal human lung stem cells (non-mhLSCs) negative for CD44/CD73/CD105 present in a pool of c-kit-positive human lung stem cells (hLSCs) are able to differentiate into alveolar epithelial cells and capillary endothelial cells. The other class of cells found in the pool of c-kit-positive human lung stem cells (hLSCs) is comprised of mesenchymal-like lung stem cells (ml-hLSCs) that are positive for CD44/CD73/CD105. non-mhLSCs negative for CD73 may have a higher ability to form lung-specific cell types, i.e., alveolar epithelial cells and capillary endothelial cells, preventing the generation of cells that would create further damage in the diseased lung. In this regard, type-1 and type 2 alveolar epithelial cells and capillary endothelial cells form the gas exchange units of the organ. Unlike the non-mhLSC clones, clonal ml-hLSCs do not acquire the epithelial and vascular cell lineages. Clonal ml-hLSCs instead differentiate into adipocytes, chondrocytes, osteocytes and fibroblasts. Notably, chronic obstructive pulmonary disease (COPD) and idiopathic or acquired pulmonary fibrosis (PF) in humans are characterized by fibroblast accumulation and tissue fibrosis. Non-mhLSC clones derived from control and diseased lung tissue displayed features of stem cell-formed colonies, while ml-hLSC clones derived from control and diseased lung tissue form non-circular clones that were characterized by cells weakly labeled for c-kit, low nucleus-to-cytoplasm ratio and positive for CD44/CD73/CD105. Importantly, the proportion of non-mhLSCs and ml-hLSCs changes significantly between control and diseased lungs, with the amount of ml-hLSCs increasing and the amount of non-mhLSCs decreasing in diseased lung tissue as compared to control healthy lung tissue. Additionally, ml-hLSCs from diseased lungs generate a large number of fibroblasts/myofibroblasts and invade the matrigel at high rate and acquire the myofibroblast phenotype. Without wishing to be bound by any theory, these data indicate that in lung diseases or disorders such as COPD, IPF or PPF, ml-hLSCs possess characteristics which make them candidates of lung pathology. With COPD, the increase in ml-hLSCs and the decrease in non-mhLSC may attenuate the ability of the COPD lung to form gas exchange units and this may lead to enlargement of alveoli, destruction of the alveolar wall and respiratory failure. By contrast, the circular clones of non-mhLSCs negative for CD44/CD73/CD105 are composed of undifferentiated cells intensely positive for c-kit, have high nucleus-to-cytoplasm ratio and are present in greater amounts in control healthy lung tissue. Thus the population of isolated non-mhLSCs from lung tissue can be transplanted or implanted into an affected/damaged lung for therapeutic purposes. The non-mhLSCs can take up residence in the lung, grow and differentiate into the various types of tissues normally found in a lung, for restoring and reconstituting the pulmonary epithelial and pulmonary vessels etc. in a damage lung, e.g., epithelial, vascular, alveolar, secretory cells, etc. The goal is to replace some of the damaged lung tissue due to disease in the affected lung. The replacement lung tissue serves to supplement existing or remaining lung tissue in the affected subject so that overall there is enough tissue for adequate gaseous exchange to sustain life in that subject.

Adult stem cell transplantation has emerged as a new alternative to stimulate repair of injured tissues and organs. In the past decade, some studies in animals and humans have documented the ability of adult bone marrow—derived stem cells, i.e., hematopoietic stem cells, to differentiate into an expanding repertoire of non-hematopoietic cell types, including brain, skeletal muscle. chondrocytes, liver, endothelium, and heart. However, the lung and associated respiratory structures have remained relatively resistant to such therapeutic modalities. There are, however, reports indicating that mesenchymal stem cells can be used for stem cell therapies in the lung, and that hematopoietic stem cells can be co-administered with mesenchymal stem cells in pulmonary transplantation. For example, it has been described that co-transplantation of mesenchymal cells, isolated as non-hematopoietic cells from fetal lung CD34+ cells, enhanced the engraftment of hematopoietic stem cells (Noort et al., Exp Hematol 2002; 30:870-78).

Several other reports also describe the use of mesenchymal stem cells and non-hematopoietic stem cells derived from bone-marrow populations in lung therapies in animal models (Krause D S et al., Cell 2001, 105:369-377; Kotton D N, et al., Development 2001, 128:5181-5188; Ortiz L A, et al., Proc Natl Acad Sci USA 2003, 100:8407-8411; Theise N D et al., Exp Hematol 2002, 30:1333-1338; Abe S et al., Cytotherapy 2003, 5:523-533; Aliotta J M et al., Exp Hematol 2006, 34:230-241; Rojas Metal., Am J Respir Cell Mol Biol 2005, 33:145-152; Gupta N et al., J Immunol 2007; 179:1855-1863; US Patent Application 20090274665).

While evidence exists supporting the ability of some types of bone marrow-derived stem cells, i.e., mesenchymal stem cells, to give rise to lung tissue, other reports have been unable to detect significant regeneration of lung tissue with bone marrow cells (Kotton D N et al., Am J Respir Cell Mol Biol 2005; 33:328-334; Wagers A J, et al., Science 2002, 297:2256-2259; Chang J C, et al. Am J Respir Cell Mol Biol 2005, 33:335-342). In addition, other reports have described that hematopoietic stem cells derived from bone marrow administered via an intranasal route results in alveolar macrophages, and that this population does not transdifferentiate into respiratory epithelial cells (Fritzell J A et al., Am J Respir Cell Mol Biol 2009, 40:575-587).

The presence of legitimate stem cells in the lung and the use of these lung stem cells (LSCs) for lung therapy are disclosed in WO 2012/047951. The advantage of the present invention is that there is a subset of the LSCs which can also be used for autologous or allogeneic lung therapy. The use of autologous cells will greatly increase success rate of the therapy. A portion of a patient's lung is removed surgically, e.g., during a biopsy. As little as one cubic centimeter is sufficient. The piece of tissue is treated to release single cells from the connective tissue. Using the stem cell marker, c-kit, as an indication of stem cells, c-kit positive cells are selected. These c-kit positive LSCs can be further negatively selected for the CD44, CD73 and CD105 markers of the mesenchymal stromal cell lineage. The non-mhLSCs are then expanded in vitro to obtain sufficient number of cells required for the therapy. When there are enough cells, the cells are harvested and injected back into the same patient or a genetically matched patient with respect to the donor of the non-mhLSCs. At each transitional step, e.g., bet between selection and expansion, or between expansion and implanting, the non-mhLSCs can be optionally cryopreserved. In one embodiment, the patient gets back the patient's own non-mhLSCs that have been selected and expanded in vitro. In another embodiment, the patient gets the non-mhLSCs derived from a genetically matched donor. In some embodiments, this method can also be extended to any mammal that has lungs, e.g., cat, dog, horse, monkey, etc.

Accordingly, the invention provides a method for treating or preventing a lung disease or disorder in a subject in need thereof, comprising: obtaining a human lung tissue from the subject in need thereof or from a different subject; extracting a population of stem cells positive for c-kit and negative for the CD44, CD73 and CD105 markers of the mesenchymal stromal cell lineage (non-mhLSCs) from said lung tissue; expanding said population of non-mhLSCs; and administering said expanded population of non-mhLSCs to the subject in need thereof.

In one embodiment, provided here is a method for treating and/or preventing a lung disease or disorder in a subject in need thereof, the method comprising administering a composition comprising a population of stem cells positive for c-kit and negative for the CD44, CD73 and CD105 markers of the mesenchymal stromal cell lineage described herein to the subject.

In another embodiment, the invention provides a method for treating or preventing a lung disease or disorder in a subject in need thereof, comprising: obtaining a human lung tissue from the subject in need thereof or from a different subject; extracting a population of stem cells positive for c-kit and negative for the CD44, CD73 and CD105 markers of the mesenchymal stromal cell lineage (non-mhLSCs) from said lung tissue; expanding said population of non-mhLSCs; and administering said expanded population of non-mhLSCs to the subject in need thereof for the repair, reconstitution or generation of pulmonary epithelium, pulmonary vasculature/pulmonary endothelium and/or pulmonary alveoli in the lungs of the subject in need thereof.

In another embodiment, the invention provides a method for treating or preventing a lung disease or disorder in a subject in need thereof, the method comprising obtaining a lung tissue from a first subject; extracting a population of stem cells positive for c-kit and negative for the CD44, CD73 and CD105 markers of the mesenchymal stromal cell lineage (non-mhLSCs) from said lung tissue; expanding said population of non-mhLSCs; and administering said expanded population of non-mhLSCs to a second subject for the non-mhLSCs to take up residence in the lungs and repair, reconstitute, and/or generate pulmonary cells and tissues in the lung of the second subject. In one embodiment of this treatment method, the second subject is at least one HLA type matched with the first subject, the donor of the non-mhLSCs.

In one embodiment of all aspects of the compositions and methods described, the non-mhLSCs that make up predominantly the population of isolated cells have self-renewal capability and clonogenicity. This means that a single isolated non-mhLSC can divide to give rise to more non-mhLSCs, forming a colony in culture. When stimulated under certain conditions, the non-mhLSC can became determinate (i.e., selection a specific cell lineage to differentiate into) and further differentiate into alveolar epithelial cells, capillary endothelial cells, or a combination thereof. These cells and its progeny, upon determination and differentiation, will express the particular cell markers characteristic of epithelial and vascular cell lineages. In addition, the determinate cell and its progeny will loss the expression of c-kit.

In one embodiment of all aspects of the compositions and methods described, the lung tissue is from a human. In another embodiment of all aspects of the compositions and methods described, the human is an adult.

In one embodiment of all aspects of the described methods, the lung tissue is cryopreserved prior to selecting non-mhLSCs.

In one embodiment of all aspects of the described methods, the selection of the non-mhLSCs is performed using an antibody against c-kit.

In one embodiment of all aspects of the described methods, the antibody against c-kit is a monoclonal antibody.

in one embodiment of all aspects of the described methods, the monoclonal antibody against c-kit is a mouse monoclonal IgG against an antigenic epitope of human c-kit.

in one embodiment of the any of the described methods, the antibody against c-kit is fluorochrome conjugated.

In one embodiment of all aspects of the described methods, the antibody against c-kit is conjugated to magnetic particles.

In one embodiment of all aspects of the described methods, the method further comprises negative selection for the CD44, CD73 and CD105 markers of the mesenchymal stromal cell lineage.

In one embodiment of all aspects of the described methods, the selection of c-kit positive cells and/or the selection of various lineage marker negative cells is by flow cytometry.

In one embodiment of all aspects of the described methods, the selection is by fluorescence activated cell sorting or high gradient magnetic selection.

In one embodiment of all aspects of the described methods, the non-mhLSCs are further expanded ex vivo. In one embodiment of all aspects of the described methods, the non-mhLSCs are further expanded in vitro. The goal is to have a sufficiently large amount of non-mhLSCs for implanting to ensure successful engrafting of the implanted non-mhLSCs into niches of the damaged lungs. Basically, there must be sufficient cells to grow and multiply in the damaged lung to provide all the cells needed to repair and/or replace the damage parts of the lungs.

In one embodiment of all aspects of the described methods, the non-mhLSCs are at least double in number after the expansion or proliferation step. In some embodiments of all aspects of the described methods, it is desirable that the number of non-mhLSCs, upon expansion or proliferation, is increased by at least 5 fold, 10 fold, 20 fold, 50 fold, 100 fold, 200 fold, 500 fold, 1000 fold, 2000 fold, 5000 fold, 10,000 fold, 20,000 fold, 50,000 fold or more at the end of the proliferation phase. The number of cells in a culture can be determined by any methods known in the art, e.g., by using a coulter counter. These methods are well known to those skilled in the art.

In one embodiment of all aspects of the described methods, the selected non-mhLSCs are cryopreserved for storage prior to expansion.

In another embodiment of all aspects of the described methods, the expanded non-mhLSCs are cryopreserved for storage purposes. When needed, the frozen cells are thawed and then used for implant into a subject in need thereof.

In one embodiment of all aspects of the described methods, the method further comprises cryopreserving the population of isolated non-mhLSCs.

For a person who has been newly diagnosed with a lung disease, if a biopsy sample of the subject's lung was obtained for the diagnosis, a population of non-mhLSCs can be prepared according to the methods described herein and the non-mhLSCs can then be cryopreserved for future use in the event that the disease had progressed to an advance stage such that the person needed a lung transplant.

Similarly, people who are at risk of developing lung diseases can benefit from early preparation of a population of non-mhLSCs form their own lung tissue and cryopreserving the non-mhLSCs. For example, a heavy smoker and a person having prior exposure to asbestos would benefit. This is because it can take anywhere from 10 to 40 years or more for symptoms of a smoking related or an asbestos-related condition to appear. Other types of people at risk of developing lung diseases or damage include, but are not limited to, a baby carrying a cystic fibrosis gene or is diagnosed with cystic fibrosis and an active military personnel deployed to a war zone.

In some embodiments of all aspects of the therapeutic methods, treating and treatment includes “restoring structural and functional integrity” to a damaged lung in a subject in need thereof.

In other embodiments of all aspects of the described methods, treating includes repairing damaged or inadequate human lung. In another embodiment, treating and treatment includes repair, reconstitution or generation of pulmonary epithelium, pulmonary vasculature/pulmonary endothelium and/or pulmonary alveoli in a damaged lung.

The restoring or repairing need not be to 100% to that of the lung of a healthy person. As long as there is an improvement in the symptoms in the subject, restoring or repairing has been achieved. A skilled physician would be able to assess the severity of the symptoms before and after the treatment and based on a comparison determine whether there is an improvement. Often, the subject will be able to say whether there is an improvement in the symptoms. Examples of some symptoms include but are limited to shortness of breath, wheezing, or hoarseness, persistent cough, pain or tightening in the chest and the presence of fluid in the lungs.

In one embodiment of all aspects of the therapeutic methods, preventing and prevention includes slowing down the reduced functioning capacity and integrity of the lung due to disease, e.g., from COPD, IPF, or PPF.

In one embodiment of all aspects of the therapeutic methods, the population of non-mhLSCs repairs, reconstitutes or generates pulmonary epithelium, pulmonary vasculature/pulmonary endothelium and/or pulmonary alveoli.

In one embodiment of all aspects of the compositions and methods described, the population of isolated non-mhLSCs is further substantially negative for the CD44, CD73 and CD105 markers of the mesenchymal stromal cell lineage.

In one embodiment of all aspects of the therapeutic methods, the method of treating and/or preventing a lung disease or disorder further comprises administering at least one therapeutic agent. Such therapeutic agent ideally would be those used for the treatment of the lung disease and these are generally known to skilled physicians, e.g., therapy for pulmonary hypertension or COPD.

In one embodiment of all aspects of the therapeutic methods, the method of treating and/or preventing a lung disease or disorder further comprises selecting a subject who is suffering from a lung disease or disorder prior to administering the population of non-mhLSCs, e.g., a subject suffering from COPD or mesothelioma.

In one embodiment of all aspects of the therapeutic methods, the method of treating and/or preventing a lung disease or disorder further comprises selecting a subject in need of restoring the structural and functional integrity of a damaged lung prior to administering the non-mhLSCs, e.g. a subject suffering from sarcoidosis.

In one embodiment of all aspects of the therapeutic methods, the method of treating and/or preventing a lung disease further comprises selecting a subject in need of treatment, prevention or repair or reconstitution or generation of pulmonary vasculature or pulmonary epithelium, pulmonary endothelium, or pulmonary alveoli prior to administering the cells, e.g., a subject suffering from pulmonary fibrosis.

For example, the selected subjects are those who have not responded at all or well to the traditional treatment and/or one who has exhausted all therapeutic options currently known in the art for a particular form or type of lung disease. Other examples of subjects to be selected would be those who are deemed not suitable subjects for any lung transplantation or who have been on the transplant waiting list for a long time without sight of a suitable donor (also there is no live donor) and is on the critical list.

In one embodiment of all aspects of the therapeutic methods for treating or preventing a lung disease, the administration is intrapulmonary administration, systemic administration, intravenous administration, or a combination thereof.

In one embodiment of all aspects of the therapeutic methods for treating or preventing a lung disease, the intrapulmonary administration is intratracheal or intranasal administration.

In one embodiment of all aspects of the therapeutic methods for treating or preventing a lung disease, the subject is an intubated subject.

In one embodiment of all aspects of the therapeutic methods for treating or preventing a lung disease, the non-mhLSCs are autologous cells.

In one embodiment of all aspects of the therapeutic methods for treating or preventing a lung disease, the non-mhLSCs are allogeneic cells obtained from one or more donors.

In one embodiment of all aspects of the therapeutic methods, the non-mhLSCs are human leukocyte antigen (HLA) typed matched for the recipient subject of the cells. In one embodiment, non-mhLSCs are isolated and expanded from a single donor and the progenitor cells are matched for at least 4 out of 6 alleles of the HLA class I: HLA-A and HLA-B; and HLA class II: DRB1 with the recipient. In another embodiment, non-mhLSCs are isolated and expanded from different donors and the progenitor cells are HLA type matched for at least 4 out of 6 alleles of the HLA class I: HLA-A and HLA-B; and HLA class II: DRB1 with the recipient subject. Methods for HLA typing are known in the art, e.g., in Bodmer, W., 1973, in Manual of Tissue Typing Techniques, Ray, J. G., et al., eds., DHEW Publication No. (NIH) 74-545, pp. 24-27 which is incorporated herein by reference in its entirety.

In one embodiment of all aspects of the therapeutic methods, the method further comprises further administering at least one therapeutic agent with the non-mhLSCs, e.g., those for treating cystic fibrosis, COPD, pulmonary fibrosis and sarcoidosis.

In one embodiment of all aspects of the therapeutic methods, the at least one therapeutic agent enhances homing, engraftment, or survival of the population of non-mhLSCs.

In one embodiment of all aspects of the therapeutic methods, the subject is a mammal, preferably a human. In another embodiment, the subject is an adult human. In one embodiment, the population of non-mhLSCs is a population of human non-mhLSCs.

### Non-mhLSCs and Ml-hLSCs in Diagnosis and Prognosis of Lung Diseases and Disorders

A pool of c-kit-positive human lung stem cells (hLSCs) comprises non-mesenchymal human lung stem cells (non-mhLSCs) negative for CD44/CD73/CD105 and mesenchymal-like lung stem cells (ml-hLSCs) that are positive for CD44/CD73/CD105. non-mhLSCs negative for CD73 may have a higher ability to form lung-specific cell types, i.e., alveolar epithelial cells and capillary endothelial cells, preventing the generation of cells that would create further damage in the diseased lung. In this regard, type-1 and type 2 alveolar epithelial cells and capillary endothelial cells form the gas exchange units of the organ. Unlike the non-mhLSC clones, clonal ml-hLSCs do not acquire the epithelial and vascular cell lineages. Clonal ml-hLSCs instead differentiate into adipocytes, chondrocytes, osteocytes and fibroblasts. Notably, chronic obstructive pulmonary disease (COPD) and idiopathic or acquired pulmonary fibrosis (PF) in humans are characterized by fibroblast accumulation and tissue fibrosis. Importantly, the proportion of non-mhLSCs and ml-hLSCs changes significantly between control and diseased lungs, with the amount of ml-hLSCs increasing and the amount of non-mhLSCs decreasing in diseased lung tissue as compared to control healthy lung tissue. Additionally, ml-hLSCs from diseased lungs generate a large number of fibroblasts/myofibroblasts and invade the matrigel at high rate and acquire the myofibroblast phenotype. Thus, ml-hLSCs possess characteristics which may make them candidates of lung pathology. With COPD, the increase in ml-hLSCs and the decrease in non-mhLSC may attenuate the ability of the COPD lung to form gas exchange units and this may lead to enlargement of alveoli, destruction of the alveolar wall and respiratory failure.

COPD is the third leading cause of death in the USA. COPD is frequently undiagnosed in its initial phases, emphasizing the need for novel diagnostic tools and new treatment strategies. COPD and PF in humans are characterized by an increase in ml-hLSCs and a decrease in non-mhLSCs. A change in the proportion of ml-hLSCs and non-mhLSCs may occur early in the process, providing an early detection of the pathologic state.

in one embodiment, another advantage of the present invention is the use of the amount of non-mhLSCs, amount of ml-hLSCs, amount of non-mhLSCs and ml-hLSCs, proportion of non-mhLSCs to ml-hLSCs, or combination thereof, to diagnose, prognose, monitor, and/or evaluate a lung disease or disorder in an individual.

In one embodiment, the disclosure provides a method of evaluating a lung disease or disorder prevalent in an affected individual compared to a healthy individual, comprising: (a) isolating non-mhLSCs and ml-hLSCs from one or more lung tissue sample from the affected individual; (b) measuring the amounts of non-mhLSCs and ml-hLSCs in the lung tissue sample obtained from said affected individual; and (c) comparing the amount of non-mhLSCs, amount of ml-hLSCs, amount of non-mhLSCs and ml-hLSCs, proportion of non-mhLSCs to ml-hLSCs, or combination thereof, to a reference value or range of reference values, wherein the reference is one or more healthy individuals, wherein a change in the amount of non-mhLSCs, amount of ml-hLSCs, amount of non-mhLSCs and ml-hLSCs, proportion of non-mhLSCs to ml-hLSCs, or combination thereof, is indicative of the lung disease or disorder prevalent in the affected individual.

In one embodiment, the disclosure provides a method of evaluating the therapeutic efficacy of a therapeutic intervention for treating a lung disease or disorder in an individual, comprising: (a) obtaining at least one initial lung tissue sample from the individual at an initial time point, wherein the initial time point is prior to the administration of the therapeutic intervention; (b) obtaining at least one subsequent lung tissue sample from the individual at a subsequent time point, wherein the subsequent time point is after the administration of the therapeutic intervention; (c) isolating non-mhLSCs and ml-hLSCs from the at least one lung tissue sample at each of said time points: (d) measuring the amounts of non-mhLSCs and ml-hLSCs in the initial and subsequent lung tissue samples; and (e) comparing the amount of non-mhLSCs, amount of ml-hLSCs, amount of non-mhLSCs and ml-hLSCs, proportion of non-mhLSCs to ml-hLSCs, or combination thereof, in the at least one initial lung tissue sample to the amount of non-mhLSCs, amount of ml-hLSCs, amount of non-mhLSCs and ml-hLSCs, proportion of non-mhLSCs to ml-hLSCs, or combination thereof, in the at least one subsequent lung tissue sample, wherein a change in the amount of non-mhLSCs, amount of ml-hLSCs, amount of non-mhLSCs and ml-hLSCs, proportion of non-mhLSCs to ml-hLSCs, or combination thereof, is indicative of the efficacy of the therapeutic intervention as a treatment for the lung disease or disorder in the individual.

In one embodiment, the disclosure provides a method of confirming or refuting a diagnosis of a lung disease or disorder in an individual, comprising: (a) isolating non-mhLSCs and ml-hLSCs from one or more lung tissue sample from the individual; (b) measuring the amounts of non-mhLSCs and ml-hLSCs in the lung tissue sample obtained from said individual; and (c) comparing the amount of non-mhLSCs, amount of ml-hLSCs, amount of non-mhLSCs and ml-hLSCs, proportion of non-mhLSCs to ml-hLSCs, or combination thereof, to a reference value or range of reference values, wherein the diagnosis of the lung disease or disorder in said individual is confirmed or refuted based on a change in the amount of non-mhLSCs, amount of ml-hLSCs, amount of non-mhLSCs and ml-hLSCs, proportion of non-mhLSCs to ml-hLSCs, or combination thereof.

In one embodiment, the disclosure provides a method of monitoring treatment of a lung disease or disorder in an individual in need thereof, comprising: (a) obtaining at least one initial lung tissue sample from the individual at an initial time point, wherein the initial time point is prior to the start of a therapeutic intervention protocol for the lung disease or disorder; (b) obtaining at least one subsequent lung tissue sample from the individual at a subsequent time point, wherein the subsequent time point is after the start of the therapeutic intervention protocol; (c) isolating non-mhLSCs and ml-hLSCs from the at least one lung tissue sample at each of said time points; (d) measuring the amounts of non-mhLSCs and ml-hLSCs in the initial and subsequent lung tissue samples; and (e) comparing the amount of non-mhLSCs, amount of ml-hLSCs, amount of non-mhLSCs and ml-hLSCs, proportion of non-mhLSCs to ml-hLSCs, or combination thereof, in the at least one initial lung tissue sample to the amount of non-mhLSCs, amount of ml-hLSCs, amount of non-mhLSCs and ml-hLSCs, proportion of non-mhLSCs to ml-hLSCs, or combination thereof, in the at least one subsequent lung tissue sample, wherein a change in the amount of non-mhLSCs, amount of ml-hLSCs, amount of non-mhLSCs and ml-hLSCs, proportion of non-mhLSCs to ml-hLSCs, or combination thereof, is indicative of the efficacy of the therapeutic intervention protocol.

In some embodiments, the lung disease or disorder is COPD, IPF, or PPF. In some embodiments, the amount of non-mhLSCs decreases in an individual having a lung disease or disorder. In another embodiment, the amount of ml-hLSCs increases in an individual having a lung disease or disorder.

### Lung Stem Cells (LSCs)

Stem cells are cells that retain the ability to renew their own kind through mitotic cell division and their daughter cells can differentiate into a diverse range of specialized cell types. The two broad types of mammalian stem cells are: embryonic stem (ES) cells that are found in blastocysts, and adult stem cells that are found in adult tissues. In a developing embryo, ESs can differentiate into all of the specialized embryonic tissues. In adult organisms, adult stem cells and progenitor cells act as a repair system for the body, replenishing specialized cells, but also maintain the normal turnover of regenerative organs, such as blood, skin or intestinal tissues. Pluripotent stem cells can differentiate into cells derived from any of the three germ layers.

In some embodiment, the term “stem cell” as used herein, refers to an undifferentiated cell which is capable of proliferation and giving rise to more progenitor cells having the ability to generate a large number of mother cells that can in turn give rise to differentiated, or differentiable daughter cells known as precursor cells. The daughter cells themselves can be induced to proliferate and produce progeny that subsequently differentiate into one or more mature cell types, while also retaining one or more cells with parental developmental potential.

in some embodiment, the term “stem cell” also refers to a subset of progenitors that have the capacity or potential, under particular circumstances, to differentiate to a more specialized or differentiated phenotype, and also retains the capacity, under certain circumstances, to proliferate without substantially differentiating.

The LSCs described herein are somatic stem cells as oppose to ESs. In a preferred embodiment, the LSCs described are adult stem cells.

In one embodiment, as used herein, the term “c-kit positive lung stem cell” or “c-kit positive LSC” encompass stem cells, progenitor cells and precursor cells, all of which are c-kit positive.

In one embodiment, as used herein, the term “c-kit positive lung stem cell” or “c-kit positive LSC” encompasses c-kit positive/KDR positive cells and c-kit positive/KDR negative cells.

In one embodiment, as used herein, the term “non-mhLSC” or “non-mesenchymal human lung stem cell” encompasses lung stem cells that are strongly c-kit positive and are CD44/CD73/CD105 negative. The non-mhLSCs can differentiate into alveolar epithelial cells, capillary endothelial cells, or a combination thereof. The non-mhLSCs are present in greater amounts in healthy control lung tissue as compared to diseased lung tissue.

In one embodiment, as used herein, the term “ml-hLSC” or “mesenchymal-like human lung stem cell” encompasses lung stem cells that are weakly c-kit positive and are CD44/CD73/CD105 positive. ml-hLSCs differentiate into adipocytes, chondrocytes, osteocytes and fibroblasts. The ml-hLSCs are present in greater amounts in diseased lung tissue as compared to healthy control lung tissue.

Cellular differentiation is a complex process typically occurring through many cell divisions. A differentiated cell may derive from a multipotent cell which itself is derived from a multipotent cell, and so on. While each of these multipotent cells may be considered stem cells, the range of cell types each can give rise to may vary considerably. Some differentiated cells also have the capacity to give rise to cells of greater developmental potential. Such capacity may be natural or may be induced artificially upon treatment with various factors. In many biological instances, stem cells are “multipotent” because they can produce progeny of more than one distinct cell type. Self-renewal is the other classical part of the stem cell definition, and it is essential as used in this document. In theory, self-renewal can occur by either of two major mechanisms. Stem cells may divide asymmetrically, with one daughter retaining the stem state and the other daughter expressing some distinct other specific function and phenotype. Alternatively, some of the stem cells in a population can divide symmetrically into two stem cells, thus maintaining some stem cells in the population as a whole, while other cells in the population give rise to differentiated progeny only.

in some embodiments, a pool of c-kit-positive human lung stem cells (hLSCs) are comprised of two cell classes: non-mesenchymal hLSCs (non-mhLSCs), that are negative for the mesenchymal epitopes CD44, CD73 and CD105; and mesenchymal-like hLSCs (ml-hLSCs), that expresses epitopes CD44, CD73 and CD105. Both cell types possess the properties of tissue specific adult stem cells, i.e., self-renewal and clonogenicity.

In one embodiment, the population of isolated cells that is substantially enriched for non-mhLSCs comprises predominantly LSCs (≥70%) and a very small amount of lung progenitor cells and lung precursor cells (≤10%). Therefore, in one embodiment, the population of isolated cells that is substantially enriched for non-mhLSCs is referred to as a population of isolated non-mhLSCs. It is meant that the population of non-mhLSCs can include some c-kit positive progenitor cells and/or c-kit precursor cells.

As used herein, in some embodiments, the term “a population of isolated and substantially enriched for non-mhLSCs”, “a population of isolated non-mhLSCs”, “population of non-mhLSCs”, “an isolated population of lung stem cells positive for c-kit and negative for the CD44, CD73 and CD105 markers of the mesenchymal stromal cell lineage”, “a population of stem cells positive for c-kit and negative for the CD44, CD73 and CD105 markers of the mesenchymal stromal cell lineage”, or “an enriched population of isolated c-kit positive lung stem cells from a human lung tissue sample wherein the c-kit positive lung stem cells are negative for the CD44, CD73 and CD105 markers of the mesenchymal stromal cell lineage” encompasses a heterogeneous or homogeneous population of non-mhLSCs and/or lung progenitor cells and/or lung precursor cells. Lung progenitor cells and lung precursor cells are lineage determinate cells. For example, if a lung progenitor cell is determinate for an epithelial lineage, i.e., will produce pulmonary epithelial cells in the future, this lung progenitor cell will not switch and produce blood cells, which are cells of the hematopoietic lineage. In some embodiments, lung progenitor cells and lung precursor cells are determinate for a pulmonary epithelial lineage, a pulmonary endothelial lineage or a pulmonary alveoli cell lineage. A population of isolated non-mhLSCs comprised of at least two different cell types is referred to herein as a “heterogeneous population”. It is also contemplated herein that lung stem cells or lung progenitor cells are isolated and expanded ex vivo prior to transplantation. A population of isolated non-mhLSCs comprising only one cell type (e.g., lung stem cells) is referred to herein as a “homogeneous population of cells”.

Lung stem cells in the human adult lung tissues express the c-kit, also called KIT or CD117, which is a cytokine receptor that binds cytokine stem cell factor (SCF). SCF signals to cells to divide and grow. In general, c-kit is expressed on the surface of stein cells as well as the progenitor and precursor cell types which are progeny from the stem cells by mitotic division. Therefore, c-kit is a stem cell marker. By immunostaining for c-kit in human adult lung tissues, the inventors found such c-kit positive cells (see WO 2012/047951). Prior to this discovery, there had been no reported evidence of the presence of stem cells in the lungs.

in one embodiment, as used herein, the term “LSC” refers to a cell with multi-lineage pulmonary differentiation potential and sustained self-renewal activity. “Self-renewal” refers to the ability of a cell to divide and generate at least one daughter cell with the identical (e.g., self-renewing) characteristics of the parent cell. The second daughter cell may commit to a particular differentiation pathway. For example, a self-renewing LSC divides and forms one daughter stem cell and another daughter cell committed to differentiation in the pulmonary epithelial or pulmonary vessel pathway. A committed progenitor cell has typically lost the self-renewal capacity, and upon cell division produces two daughter cells that display a more differentiated (i.e., restricted) phenotype.

“LCSs,” as used in the methods described herein, therefore, encompasses all pluripotent cells capable of differentiating into several cell types of the respiratory system, including, but not limited to, pneumocyte type 1 and type II cells, interalveolar cells, smooth muscle cells, alveoli epithelial cells, endothelial cells and erythrocytes.

“Lung progenitor cells,” as the term is used herein, refer to the subset of LSC that are committed to a particular pulmonary cell lineage and generally do not self-renew, and can be identified, for example by cell surface markers or intracellular proteins. For example, TTF1 which indicates commitment to the pulmonary epithelial lineage; or GATA6 and/or Est1 which indicates commitment to the pulmonary vessel lineage.

The presence of non-mhLSCs and/or ml-hLSCs can be determined by any method known in the art, or phenotypically through the detection of cell surface markers using assays known to those of skill in the art or those described in the example.

### Isolation of LSCs

In some embodiments of all aspects of the compositions and methods described, the non-mhLSCs and/or ml-hLSCs are derived or isolated from lung tissue samples of the following sources: aborted fetus, fetal biopsy tissue, freshly deceased subjects, tissue biopsy from a live subject, a lung stem cell line. In some embodiments of all aspects of the compositions and methods described, the non-mhLSCs and/or ml-hLSCs am derived ex vivo from other cells, such as embryonic stem cells, induced pluripotent stem cells (iPS cells) or adult pluripotent cells.

In one embodiment of all aspects of the compositions and methods described, the non-mhLSCs can be isolated using any method known to one of skill in the art or according to the method described herein. For example, fine needle aspiration from a small lung tissue sample from a live subject.

Non-mhLSCs and/or ml-hLSCs can be isolated from lung tissue samples by any method known in the art. Methods of dissociating individual cells from a tissue sample are known in the art, e.g., in U.S. Pat. No. 7,547,674 and U. S. Patent Application U. S. 2006/0239983, 2009/0148421, and 2009/0180998. These references are herein incorporated by reference in their entireties.

in one embodiment of all aspects of the compositions and methods described, the population of isolated non-mhLSCs is isolated by the following method. One skilled in the art would be able to make minor adjustment to the method as needed for lung tissues from different sources. A small piece of lung tissue, a minimum size of at least 1 cubic cm, is enzymatically digested with collagenase to obtain single cells (Kajstura, J., et al., 2011, New Engl J Med 364: 1795-1806). Small intact cells are resuspended and aggregates of cells are removed with a cell strainer. This cell strainer step is optional. Then the cells are incubated with a mouse c-kit antibody. Single c-kit positive cells are isolated and collected with immunomagnetic beads coated with anti-mouse IgG. non-mhLSCs are further selected by negative selection of the CD44/CD73/CD105 markers.

In one embodiment of all aspects of the compositions and methods described, the isolated non-mhLSCs obtained are then cultured by the following method. One skilled in the art would be able to make minor adjustment to the method as needed. The culture method is used to grow and expand the number of non-mhLSCs. The isolated non-mhLSCs are plated in modified F12K medium containing F12 medium (GIBCO, Grand island, NY) supplemented with 5-10% FBS (GIBCO) and insulin-selenium-transferrin mixture (SIGMA, St. Louis, Mo.) under standard tissue culture conditions. After reaching confluence, the cells are passaged to several other plates to expand the culture using standard tissue culture protocol of handling the cells.

In some embodiments of all aspects of the compositions and methods described, the non-mhLSCs from the lung tissues described herein is expanded ex vivo using any method acceptable to those skilled in the art prior to use in the methods described herein. In some embodiments of all aspects of the compositions and methods described, the expanded non-mhLSCs are further sorted, fractionated, treated to remove any undesired cells, or otherwise manipulated to treat the patient using any procedure acceptable to those skilled in the art of preparing cells for transplantation. An example of an undesired cell is a malignant cell.

There is typically a very small number of non-mhLSCs in a sample of lung tissue, for example, there can be only one or two non-mhLSCs per one million cells. Therefore, expansion of the selected non-mhLSCs is necessary to increase the number of cells required for the therapeutic uses described herein. The greater number of non-mhLSCs transplanted in the therapeutic uses described herein increases the success rate of the therapy used therein. The non-mhLSCs are used to repair, reconstitute and generate some of the damaged tissues and cells in the subject's lung. Therefore, more non-mhLSCs transplanted means more cells available to repair, reconstitute and generate new lung cells and lung tissue. In some embodiments, a success of the transplant therapy can be measured by any method known in the art and those described herein, such as an improvement in the subject's lung function, blood oxygen saturation and general health conditions which are known to a physician skilled in the art.

In some embodiments of all aspects of the compositions and methods described, a lung tissue sample comprising LSC is isolated from a subject and is then further processed, for example, by cell sorting (e.g., FACS), to obtain a population of substantially enriched non-mhLSCs. In other embodiments of all aspects of the compositions and methods described, a population of substantially enriched non-mhLSCs refers to an in vitro or ex vivo culture of expanded non-mhLSCs.

In some embodiments of all aspects of the compositions and methods described, the lung tissue samples from the various sources are frozen samples, such as frozen or cryopreserved prior to extraction or selection of the non-mhLSCs. The lung tissue sample is obtained from a subject or other sources described herein and then cryopreserved with cryoprotectant. In another embodiment of all aspects of the compositions and methods described, the population of isolated non-mhLSCs from the lung tissue sample is cryopreserved with cryoprotectant prior to use. In yet another embodiment of all aspects of the compositions and methods described, the population of isolated non-mhLSCs that has been expanded in vitro culture is cryopreserved with cryoprotectant prior to use. Methods of cryopreservation of tissues and cells with cryoprotectant are well known in the art. Further methods for thawing the cryopreserved tissue or cells for use are also well known in the art.

The terms “isolate” and “methods of obtaining or preparing,” as used herein, refer to a process whereby a cell or a population of cells, such as a population of non-mhLSCs, is removed from a subject or a lung tissue sample in which it was originally found. The term “isolated population,” as used herein, refers to a population of cells that has been removed and separated from a biological sample, or a mixed or heterogeneous population of cells found in such a sample. Such a mixed population includes, for example, a population of non-mhLSCs obtained from a lung tissue sample. In some embodiments, an isolated population is a substantially pure population of cells as compared to the heterogeneous population from which the cells were isolated or enriched from. In some embodiments, the isolated population is a population of isolated non-mhLSCs. In other embodiments of this aspect and all aspects described herein, the isolated population comprises a substantially enriched population of non-mhLSCs s. In some embodiments, an isolated cell or cell population, such as a population of non-mhLSCs, is further cultured in vitro or ex vivo, e.g., in the presence of growth factors or cytokines, to further expand the number of cells in the isolated cell population or substantially non-mhLSC enriched cell population. Such culture can be performed using any method known to one of skill in the art. In some embodiments, the isolated or substantially enriched non-mhLSC populations obtained by the methods disclosed herein are later administered to a second subject, or re-introduced into the subject from which the cell population was originally isolated (e.g., allogeneic transplantation vs. autologous administration).

The term “substantially enriched,” with respect to a particular cell population, refers to a population of cells that is at least about 70%, at least about 75%, at least about 80%, at least about 85%, at least about 90%, at least about 95%, at least about 98%, or at least about 99% pure, with respect to the cells making up a total cell population. In other words, the terms “substantially enriched” or “essentially purified”, with regard to a population of non-mhLSCs isolated for use in the methods disclosed herein, refers to a population of non-mhLSCs that contain fewer than about 25%, fewer than about 20%, fewer than about 15%, fewer than about 10%, fewer than about 9%, fewer than about 8%, fewer than about 7%, fewer than about 6%, fewer than about 5%, fewer than about 4%, fewer than about 3%, fewer than about 2%, fewer than about 1%, or less than 1%, of cells that are not non-mhLSC, as defined by the terms herein. Some embodiments of these aspects further encompass methods to expand a population of substantially pure or enriched non-mhLSCs, wherein the expanded population of non-mhLSCs is also a substantially pure or enriched population of non-mhLSCs.

The term “substantially negative,” with respect to a particular marker presence in a cell population, refers to a population of cells that is not more than about 1%, not more than about 0.9%, not more than about 0.8%, not more than about 0.7%, not more than about 0.6%, not more than about 0.5%, not more than about 0.4%, not more than about 0.3%, not more than about 0.2%, or not more than about 0.1% positive for that marker, with respect to the cells making up a total cell population.

The terms “enriching” or “enriched” are used interchangeably herein and mean that the yield (fraction) of cells of one type, such as non-mhLSCs for use in the methods described herein, is increased by at least 15%, by at least 20%, by at least 25%, by at least 30%, by at least 35%, by at least 40%, by at least 45%, by at least 50%, by at least 55%, by at least 60%, by at least 65%, by at least 70%, or by at least 75%, over the fraction of cells of that type in the starting biological sample, culture, or preparation. A population of non-mhLSCs obtained for use in the methods described herein is most preferably at least 60% enriched for non-mhLSCs.

In some embodiments, markers specific for non-mhLSCs are used to isolate or enrich for these cells. A “marker,” as used herein, describes the characteristics and/or phenotype of a cell. Markers can be used for selection of cells comprising characteristics of interest. Markers will vary with specific cells. Markers are characteristics, whether morphological, functional or biochemical (enzymatic), particular to a cell type, or molecules expressed by the cell type. Preferably, such markers are proteins, and more preferably, possess an epitope for antibodies or other binding molecules available in the art. However, a marker may consist of any molecule found in a cell including, but not limited to, proteins (peptides and polypeptides), lipids, polysaccharides, nucleic acids and steroids. Examples of morphological characteristics or traits include, but are not limited to, shape, size, appearance (e.g., smooth, translucent), and nuclear to cytoplasmic ratio. Examples of functional characteristics or traits include, but are not limited to, the ability to adhere to particular substrates, ability to incorporate or exclude particular dyes, ability to migrate under particular conditions, and the ability to differentiate along particular lineages. Markers may be detected by any method available to one of skill in the art.

Accordingly, as used herein, a “cell-surface marker” refers to any molecule that is expressed on the surface of a cell. Cell-surface expression usually requires that a molecule possesses a transmembrane domain. Some molecules that are normally not found on the cell-surface can be engineered by recombinant techniques to be expressed on the surface of a cell. Many naturally occurring cell-surface markers are termed “CD” or “cluster of differentiation” molecules. Cell-surface markets often provide antigenic determinants to which antibodies can bind to. A cell-surface marker of particular relevance to the methods described herein is CD117 or c-kit. The useful non-mhLSCs according to the compositions and method preferably express c-kit or in other words, they are c-kit positive.

A cell can be designated “positive” or “negative” for any cell-surface marker or other intracellular marker, and both such designations are useful for the practice of the methods described herein. A cell is considered “positive” for a cell-surface marker if it expresses the marker on its cell-surface or intracellularly in amounts sufficient to be detected using methods known to those of skill in the art, such as contacting a cell with an antibody that binds specifically to that marker, and subsequently performing flow cytometric analysis of such a contacted cell to determine whether the antibody is bound the cell. It is to be understood that while a cell can express messenger RNA for a cell-surface marker, in order to be considered positive for the methods described herein, the cell must express it on its surface. Similarly, a cell is considered “negative” for a cell-surface marker or other intracellular marker if it does not express the marker in amounts sufficient to be detected using methods known to those of skill in the art, such as contacting a cell with an antibody that binds specifically to that marker and subsequently performing flow cytometric analysis of such a contacted cell to determine whether the antibody is bound the cell.

In some embodiments of all aspects of the compositions and methods described, the non-mhLSCs are negatively selected and the selection uses an agent specific for a cell surface marker. In some embodiments of all aspects of the compositions and methods described, the cell surface marker is a mesenchymal stromal cell lineage marker.

In some embodiments of all aspects of the compositions and methods described, in the context of negative selection, where agents specific for lineage markers are used, all of the agents can comprise the same label or tag, such as fluorescent tag, and thus all cells positive for that label or tag can be excluded or removed, leaving the lineage marker-negative non-mhLSCs, lung progenitor cells and/or lung precursor cells for use in the methods described herein. This is negative selection, selecting for those cells that did not contact with the agents specific for lineage markers.

Accordingly, as defined herein, an “agent specific for a cell-surface marker or other intracellular marker” refers to an agent that can selectively react with or bind to that cell-surface marker or other intracellular marker, but has little or no detectable reactivity to another cell-surface marker, other intracellular marker or antigen. For example, an agent specific for c-kit will not identify or bind to CD49e. Thus, agents specific for cell-surface markers or other intracellular marker recognize unique structural features of the markers. In some embodiments, an agent specific for a marker binds to the marker, but does not cause initiation of downstream signaling events mediated by that marker, for example, a non-activating antibody. Agents specific for cell-surface molecules include, but are not limited to, antibodies or antigen-binding fragments thereof, natural or recombinant ligands, small molecules, nucleic acid sequence and nucleic acid analogues, intrabodies, aptamers, and other proteins or peptides.

In some embodiments of all aspects of the compositions and methods described, the preferred agents specific for cell-surface markers used for isolating non-mhLSCs are antibody agents that specifically bind the cell-surface markers, and can include polyclonal and monoclonal antibodies, and antigen-binding derivatives or fragments thereof. Well-known antigen binding fragments include, for example, single domain antibodies (dAbs; which consist essentially of single VL or VH antibody domains), Fv fragment, including single chain Fv fragment (scFv), Fab fragment, and F(ab′)2 fragment. Methods for the construction of such antibody molecules are well known in the art. Accordingly, as used herein, the term “antibody” refers to an intact immunoglobulin or to a monoclonal or polyclonal antigen-binding fragment with the Fc (crystallizable fragment) region or FcRn binding fragment of the Fc region. Antigen-binding fragments may be produced by recombinant DNA techniques or by enzymatic or chemical cleavage of intact antibodies. “Antigen-binding fragments” include, inter alia, Fab, Fab′, F(ab′)2, Fv, dAb, and complementarity determining region (CDR) fragments, single-chain antibodies (scFv), single domain antibodies, chimeric antibodies, diabodies and polypeptides that contain at least a portion of an immunoglobulin that is sufficient to confer specific antigen binding to the polypeptide. The terms Fab, Fc, pFc′, F(ab′) 2 and Fv are employed with standard immunological meanings known to those skilled in the art, e.g., in Klein, “Immunology” (John Wiley, New York, N.Y., 1982); Clark, W. R. (1986); in “The Experimental Foundations of Modern Immunology” (Wiley & Sons, Inc., New York); and Roitt, I. (1991) “Essential immunology”, 7th Ed., (Blackwell Scientific Publications, Oxford). Such antibodies or antigen-binding fragments are available commercially from vendors such as R&D Systems, BD Biosciences, e-Biosciences and Miltenyi, or can be raised against these cell-surface markers or other intracellular marker by methods known to those skilled in the art.

In some embodiments of all aspects of the compositions and methods described, an agent specific for a cell-surface molecule or other intracellular marker, such as an antibody or antigen-binding fragment, is labeled with a tag to facilitate the isolation of the lung stem cells. The terms “label” or “tag”, as used herein, refer to a composition capable of producing a detectable signal indicative of the presence of a target, such as, the presence of a specific cell-surface marker in a biological sample. Suitable labels include fluorescent molecules, radioisotopes, nucleotide chromophores, enzymes, substrates, chemiluminescent moieties, magnetic particles, bioluminescent moieties, and the likes. As such, a label is any composition detectable by spectroscopic, photochemical, biochemical, immunochemical, electrical, optical or chemical means needed for the methods to isolate and enrich for LSCs, lung progenitor cell and lung precursor cells.

The terms “labeled antibody” or “tagged antibody”, as used herein, includes antibodies that are labeled by detectable means and include, but are not limited to, antibodies that are fluorescently, enzymatically, radioactively, and chemiluminescently labeled. Antibodies can also be labeled with a detectable tag, such as c-Myc, HA, VSV-G, HSV, FLAG, V5, or HIS, which can be detected using an antibody specific to the tag, for example, an anti-c-Myc antibody. Various methods of labeling polypeptides and glycoproteins are known in the art and may be used. Non-limiting examples of fluorescent labels or tags for labeling the antibodies for use in the methods of invention include hydroxycoumarin, succinimidyl ester, aminocoumarin, succinimidyl ester, methoxycoumarin, Cascade Blue, Hydrazide, Pacific Blue, maleimide, Pacific Orange, lucifer yellow, NBD, NBD-X, R-phycoerythrin (PE), a PE-Cy5 conjugate (Cychrome, R670, Tri-Color, Quantum Red), a PE-Cy7 conjugate, Red 613, PE-Texas Red, PerCP, Peridinin chlorphyll protein, TruRed (PerCP-Cy5.5 conjugate), FluorX, Fluoresceinisothyocyanate (FITC), BODIPY-FL, TRITC, X-Rhodamine (XRITC), Lissamine Rhodamine B, Texas Red, Allophycocyanin (APC), an APC-Cy7 conjugate, ALEXA FLUOUR® 350, ALEXA FLUOUR® 405, ALEXA FLUOUR® 430, ALEXA FLUOUR® 488, ALEXA FLUOR® 500, ALEXA FLUOUR® 514, ALEXA FLUOUR® 532, ALEXA FLUOUR® 546, ALEXA FLUOUR® 555, ALEXA FLUOUR® 568, ALEXA FLUOUR® 594, ALEXA FLUOUR® 610, ALEXA FLUOUR® 633, ALEXA FLUOR® 647, ALEXA FLUOUR® 660, ALEXA FLUOUR® 680, ALEXA FLUOUR® 700, ALEXA FLUOUR® 750, ALEXA FLUOUR® 790, Cy2, Cy3, Cy3B, Cy3.5, Cy5, Cy5.5 or Cy7.

In some embodiments of all aspects of the compositions and methods described, a variety of methods to isolate a substantially pure or enriched population of non-mhLSCs are available to a skilled artisan, including immunoselection techniques, such as high-throughput cell sorting using flow cytometric methods, affinity methods with antibodies labeled to magnetic beads, biodegradable beads, non-biodegradable beads, and antibodies panned to surfaces including dishes and combination of such methods.

In some embodiments of all aspects of the compositions and methods described, the isolation and enrichment for populations of non-mhLSCs can be performed using bead based sorting mechanisms, such as magnetic beads. In such methods, a digested lung tissue sample is contacted with magnetic beads coated with antibodies against one or more specific cell-surface antigens, such as c-kit. This causes the cells in the sample that express the respective antigen to attach to the magnetic beads. After a period of time to allow the c-kit positive cells bind the beads, the mixture of cell and beads are exposed to a strong magnetic field, such as a column or rack having a magnet. The cells attached to the beads (expressing the cell-surface marker) stay on the column or sample tube, while other cells (not expressing the cell-surface marker) flow through or remain in solution. Using this method, cells can be separated positively or negatively, or using a combination therein, with respect to the particular cell-surface markers.

In some embodiments of all aspects of the compositions and methods described, magnetic activated cell sorting (MACS) strategies are used for isolation and pre-selection of non-mhLSCs. In some embodiments, non-mhLSCs are isolated in the presence of human plasma or human serum albumin (HSA), such as 2% HSA.

In some preferred embodiments of all aspects of the compositions and methods described, non-mhLSCs and/or ml-hLSCs are isolated or enriched using positive selection for the cell-surface marker c-kit.

In other embodiments of all aspects of the compositions and methods described, one or more additional cell-surface markers are used for isolating and/or enriching for non-mhLSCs, using positive or negative selection methods, or a combination therein. Such additional cell-surface markers include CD44, CD73 and CD105.

As defined herein, “positive selection” refers to techniques that result in the isolation or enrichment of cells expressing specific cell-surface markers or intracellular proteins, while “negative selection” refers techniques that result in the isolation or enrichment of cells that do not expressing specific cell-surface markers or intracellular proteins. Negative selection can be performed by any method known in the art. For example, typical negative selection is carried out by removing the cells that do express the marker of interest.

In some embodiments of all aspects of the compositions and methods described, beads can be coated with antibodies by a skilled artisan using standard techniques known in the art, such as commercial bead conjugation kits. In some embodiments, a negative selection step is performed to remove cells expressing one or more lineage markers, followed by fluorescence activated cell sorting to positively select ml-hLSCs expressing one or more specific cell-surface markers. For example, in a negative selection protocol, a digested lung tissue sample, is first contacted with labeled antibodies specific for cell-surface markers of interest, such as CD2, CD3, CD6, CD8, CD34, CD49e, and CD66b and the sample is then contacted with beads that are specific for the labels of the antibodies, and the cells expressing the markers CD2, CD3, CD6, CD8, CD34, CD49e, and CD66b are removed using immunomagnetic lineage depletion.

A number of different cell-surface markers have specific expression on specific differentiated cell lineages, and are not expressed by the non-mhLSCs isolated for the methods described herein. Accordingly, when agents specific for these lineage cell-markers are contacted with non-mhLSCs, the cells will be “negative.” Lineage cell-markers that are not expressed by the non-mhLSCs described herein are CD44, CD73 and CD105 (for mesenchymal stromal cell lineage).

in some embodiments of all aspects of the compositions and methods described, flow cytometric methods, alone or in combination with magnetic bead based methods, are used to isolate or enrich for non-mhLSCs. As defined herein, “flow cytometry” refers to a technique for counting and examining microscopic particles, such as cells and chromosomes, by suspending them in a stream of fluid and passing them through an electronic detection apparatus. Flow cytometry allows simultaneous multiparametric analysis of the physical and/or chemical parameters of up to thousands of particles per second, such as fluorescent parameters. Modern flow cytometric instruments usually have multiple lasers and fluorescence detectors. Increasing the number of lasers and detectors allows for labeling by multiple antibodies, and can more precisely identify a target population by their phenotypic markers. Certain flow cytometric instruments can take digital images of individual cells, allowing for the analysis of fluorescent signal location within or on the surface of cells.

A common variation of flow cytometric techniques is to physically sort particles based on their properties, so as to purify populations of interest, using “fluorescence-activated cell sorting” As defined herein, “fluorescence-activated cell sorting” or “flow cytometric based sorting” methods refer to flow cytometric methods for sorting a heterogeneous mixture of cells from a single biological sample into one or more containers, one cell at a time, based upon the specific light scattering and fluorescent characteristics of each cell and provides fast, objective and quantitative recording of fluorescent signals from individual cells as well as physical separation of cells of particular interest. Accordingly, in those embodiments when the agents specific for cell-surface markers are antibodies labeled with tags that can be detected by a flow cytometer, fluorescence-activated cell sorting (FACS) can be used in and with the methods described herein to isolate and enrich for populations of LSCs.

### Expansion of Non-mhLSCs

In some embodiments of all aspects of the compositions and methods described, the population of isolated and substantially enriched non-mhLSCs is further expanded to increase in numbers prior to their use in the therapeutic methods described herein.

In some embodiments of all aspects of the compositions and methods described, non-mhLSCs isolated or enriched by using the methods and techniques described herein are expanded in culture, i.e., the cell numbers are increased outside the body of the subject, using methods known to one of skill in the art, prior to administration to a subject in need.

In one embodiment of all aspects of the compositions and methods described, the isolated non-mhLSCs obtained are expanded in culture according to the following method. One skilled in the art would be able to make minor adjustment to the method as needed. The isolated non-mhLSCs are plated in modified F12K medium containing F12 medium (GIBCO, Grand Island, N.Y.) supplemented with 5-10% FBS (GIBCO) and insulin-selenium-transferrin mixture (SIGMA, St. Louis, Mo.) under standard tissue culture conditions, e.g., 95% air, 5% CO₂, 37° C. After reaching confluence, the cells from one confluent plate are passaged to several other plates to expand the culture using standard tissue culture protocol of handling the cells.

in some embodiments of all aspects of the compositions and methods described, such expansion methods can comprise, for example, culturing the non-mhLSCs in serum-free medium supplemented with factors and/or under conditions that cause expansion of LSCs, such as stem cell factor, IL-3, and GM-CSF. In some embodiments of all aspects of the compositions and methods described, the non-mhLSCs can further be cultured with factors and/or under conditions aimed at inducing differentiation of the LSCs to respiratory epithelia, such as using small airway growth medium, modified mouse tracheal epithelial cell medium, or serum-free medium supplemented with retinoic acid and/or keratinocyte growth factor.

In other embodiments of all aspects of the compositions and methods described, non-mhLSCs are expanded by adapting not more than about 0.5%, nanotechnological or nanoengineering methods, as reviewed in Lu J et al., “A Novel Technology for Hematopoietic Stem Cell Expansion using Combination of Nanofiber and Growth Factors.” Recent Pat Nanotechnol. 2010 4(2):125-35. For example, in some embodiments, nanoengineering of stem cell microenvironments can be performed. As used herein, secreted factors, stem cell—neighboring cell interactions, extracellular matrix (ECM) and mechanical properties collectively make up the “stem cell microenvironment”. Stem cell microenvironment nanoengineering can comprise the use of micro/nanopatterned surfaces, nanoparticles to control release growth factors and biochemicals, nanofibers to mimic extracellular matrix (ECM), nanoliter-scale synthesis of arrayed biomaterials, self-assembly peptide system to mimic signal clusters of stem cells, nanowires, laser fabricated nanogrooves, and nanophase thin films to expand LSCs.

In other embodiments of all aspects of the compositions and methods described, the non-mhLSCs are genetically manipulated, e.g., transfected with an exogenous nucleic acid. Nanoengineering can be used for the transfection and genetic manipulation in LSCs, such as nanoparticles for in vivo gene delivery, nanoneedles for gene delivery to LSCs, self-assembly peptide system for LSC transfection, nanowires for gene delivery to LSCs, and micro/nanofluidic devices for LSC electroporation.

In other embodiments of all aspects of the compositions and methods described, the non-mhLSCs isolated or enriched for use in the methods can be expanded using bioreactors.

The terms “increased,” “increase” or “expand”, when used in the context of non-mhLSCs expansion, generally mean an increase in the number of non-mhLSCs by a statistically significant amount; for the avoidance of any doubt, the terms “increased,” “increase,” “expand” or “expanded,” mean an increase, as compared to a reference level, of at least about 10%, of at least about 15%. of at least about 20%, of at least about 25%, of at least about 30%, of at least about 35%, of at least about 40%, of at least about 45%, of at least about 50%, of at least about 55%, of at least about 60%, of at least about 65%, of at least about 70%, of at least about 75%, of at least about 80%, of at least about 85%, of at least about 90%, of at least about 95%, or up to and including a 100%, or at least about a 2-fold, or at least about a 3-fold, or at least about a 4-fold, or at least about a 5-fold, at least about a 6-fold, or at least about a 7-fold, or at least about a 8-fold, at least about a 9-fold, or at least about a 10-fold increase, or any increase of 10-fold or greater, as compared to a control or reference level. A control/reference sample or level is used herein to describe a population of cells obtained from the same biological source that has, for example, not been expanded using the methods described herein, e.g., at the start of the expansion culture or the initial number of cells added to the expansion culture.

Storage of Lung Tissue Samples and/or Lung Stem Cells

In some embodiments of all aspects of the compositions and methods described, the lung tissue samples are stored prior to use, i.e., prior to the extraction, isolation or selection of the non-mhLSCs therein. In some embodiments of all aspects of the compositions and methods described, the digested lung tissue sample is stored prior to extraction or selection of the non-mhLSCs therein. In some embodiments of all aspects of the compositions and methods described, the isolated non-mhLSCs are stored. In other embodiments of all aspects of the compositions and methods described, the non-mhLSCs are first isolated and/or expanded prior to storage. In one embodiment, the storage is by cryopreservation. The non-mhLSCs are thawed when needed for the therapeutic methods described herein.

In some embodiments of all aspects of the compositions and methods described, the lung tissue samples or isolated non-mhLSCs (expanded or otherwise) are frozen prior to their use in the methods described herein. Freezing the samples can be performed in the presence of one or more different cryoprotectants for minimizing cell damage during the freeze-thaw process. For example, dimethyl sulfoxide (DMSO), trehalose, or sucrose can be used.

### Administration and Uses of Non-mhLSCs in Regenerative Medicine

Certain embodiments described herein are based on the discovery of non-mesenchymal human lung stem cells (non-mhLSCs) negative for CD44/CD73/CD105 present in a pool of c-kit-positive human lung stem cells (hLSCs) that are able to differentiate into alveolar epithelial cells and capillary endothelial cells. non-mhLSCs negative for CD73 may have a higher ability to form lung-specific cell types, i.e., alveolar epithelial cells and capillary endothelial cells, preventing the generation of cells that would create further damage in the diseased lung. In this regard, type-1 and type 2 alveolar epithelial cells and capillary endothelial cells form the gas exchange units of the organ. These observations indicated that isolated non-mhLSCs can be used for pulmonary vascular regeneration and alveolar development.

Accordingly, provided herein are methods for the treatment and/or prevention of a respiratory/lung disease or disorder in a subject in need thereof. As used herein, the term “respiratory disease or disorder” “lung disease or disorder” and “lung disorder” are used interchangeably. Some of these methods involve administering to a subject a therapeutically effective amount of isolated non-mhLSCs using intrapulmonary administration, such as an intranasal, intratracheal or intravenous route. In some aspects of these methods, a therapeutically effective amount of isolated non-mhLSCs is administered using a systemic, such as an intraperitoneal or intravenous route. In other aspects of these methods, a therapeutically effective amount of isolated non-mhLSCs is administered using both intrapulmonary and intraperitoneal administration. These methods are particularly aimed at therapeutic and prophylactic treatments of human subjects having or at risk for a respiratory disease or disorder, e.g., a subject having COPD. The isolated or enriched non-mhLSCs described herein can be administered to a selected subject having any respiratory disease or disorder or is predisposed to developing one, the administration can be by any appropriate route which results in an effective treatment in the subject. In some embodiments of all aspects of the therapeutic methods described herein, a subject having a respiratory disorder is first selected prior to administration of the cells.

The terms “subject”, “patient” and “individual” are used interchangeably herein, and refer to an animal, for example, a human from whom cells for use in the methods described herein can be obtained (i.e., donor subject) and/or to whom treatment, including prophylactic treatment, with the cells as described herein, is provided, i.e., recipient subject. For treatment of those conditions or disease stales that are specific for a specific animal such as a human subject, the term subject refers to that specific animal. The “non-human animals” and “non-human mammals” as used interchangeably herein, includes mammals such as rats, mice, rabbits, sheep, cats, dogs, cows, pigs, and non-human primates. The term “subject” also encompasses any vertebrate including but not limited to mammals, reptiles, amphibians and fish. However, advantageously, the subject is a mammal such as a human, or other mammals such as a domesticated mammal, e.g., dog, cat, horse, and the like, or food production mammal, e.g., cow, sheep, pig, and the like.

Accordingly, in some embodiments of the therapeutic methods described herein, a subject is a recipient subject, i.e., a subject to whom the isolated non-mhLSCs are being administered, or a donor subject, i.e., a subject from whom a lung tissue sample comprising non-mhLSCs are being obtained. A recipient or donor subject can be of any age. In some embodiments, the subject is a “young subject,” defined herein as a subject less than 10 years of age. In other embodiments, the subject is an “infant subject,” defined herein as a subject is less than 2 years of age. In some embodiments, the subject is a “newborn subject,” defined herein as a subject less than 28 days of age. In one embodiment, a young, infant or newborn recipient or donor subject is a non-adult recipient or donor subject. In one embodiment, a subject who is greater than 10 years of age but not an adult is a non-adult subject. In some embodiments, the recipient or donor subject is a non-adult. In a preferred embodiment, the subject is a human adult.

In some embodiments of the therapeutic methods described herein, the isolated non-mhLSCs population being administered comprises allogeneic non-mhLSCs obtained from one or more donors. As used herein, “allogeneic” refers to non-mhLSCs or lung tissue samples comprising non-mhLSCs obtained from one or more different donors of the same species, where the genes at one or more loci are not identical. For example, an isolated non-mhLSCs population being administered to a subject can be obtained from the lung tissue obtained from one more unrelated donor subjects, or from one or more non-identical siblings or other sources. In some embodiments, syngeneic isolated non-mhLSC populations are used, such as those obtained from genetically identical animals, or from identical twins. In other embodiments of this aspect, the isolated non-mhLSCs are autologous non-mhLSCs. As used herein, “autologous” refers to non-mhLSCs or lung tissue samples comprising non-mhLSCs obtained or isolated from a subject and being administered to the same subject, i.e., the donor and recipient are the same.

Lung disease is any disease or disorder that occurs in the lungs or that causes the lungs to not work properly. There are three main types of lung disease. Most lung diseases actually involve a combination of these categories: (1) Airway diseases—These diseases affect the tubes (airways) that carry oxygen and other gases into and out of the lungs. These diseases cause a narrowing or blockage of the airways. They include asthma, emphysema, and chronic bronchitis. People with airway diseases sometimes describe the feeling as “trying to breathe out through a straw.” (2) Lung tissue diseases—These diseases affect the structure of the lung tissue. Scarring or inflammation of the tissue makes the lungs unable to expand fully (“restrictive lung disease”). It also makes the lungs less capable of taking up oxygen (oxygenation) and releasing carbon dioxide. Pulmonary fibrosis and sarcoidosis are examples of lung tissue diseases. People sometimes describe the feeling as “wearing a too-tight sweater or vest” that won't allow them to take a deep breath. (3) Pulmonary circulation diseases—These diseases affect the blood vessels in the lungs. They are caused by clotting, scarring or inflammation of the blood vessels in the lungs. They affect the ability of the lungs to take up oxygen and to release carbon dioxide. These diseases can also affect heart function.

The most common lung diseases include: asthma, chronic bronchitis, chronic obstructive pulmonary disease (COPD), emphysema, pulmonary fibrosis and sarcoidosis. Other lung diseases include: asbestosis, aspergilloma, aspergillosis, acute invasive atelectasis, eosinophilic pneumonia, lung cancer, metastatic lung cancer, necrotizing pneumonia, pleural effusion pneumoconiosis, pneumocystosis, pneumonia, pneumonia in immunodeficient patient, pneumothorax, pulmonary actinomycosis, pulmonary alveolar proteinosis, pulmonary anthrax, pulmonary arteriovenous malformation, pulmonary edema, pulmonary embolus, pulmonary histiocytosis X (eosinophilic granuloma), pulmonary hypertension, pulmonary nocardiosis, pulmonary tuberculosis, pulmonary veno-occlusive disease, and rheumatoid lung disease.

The methods described herein can be used to treat, ameliorate the symptoms, prevent and/or slow the progression of a number of respiratory diseases or their symptoms, such as those resulting in pathological damage to lung or airway architecture and/or alveolar damage. The terms “respiratory disorder,” “respiratory disease,” “pulmonary disease,” and “pulmonary disorder,” are used interchangeably herein and refer to any condition and/or disorder relating to respiration and/or the respiratory system, including the lungs, pleural cavity, bronchial tubes, trachea, upper respiratory tract, airways, or other components or structures of the respiratory system. Such respiratory diseases include, but are not limited to, bronchopulmonary dysplasia (BPD), chronic obstructive pulmonary disease (COPD) condition, cystic fibrosis, bronchiectasis, cor pulmonale, pneumonia, lung abscess, acute bronchitis, chronic bronchitis, emphysema, pneumonitis, e.g., hypersensitivity pneumonitis or pneumonitis associated with radiation exposure, alveolar lung diseases and interstitial lung diseases, environmental lung disease (e.g., associated with asbestos, fumes or gas exposure), aspiration pneumonia, pulmonary hemorrhage syndromes, amyloidosis, connective tissue diseases, systemic sclerosis, ankylosing spondylitis, pulmonary actinomycosis, pulmonary alveolar proteinosis, pulmonary anthrax, pulmonary edema, pulmonary embolus, pulmonary inflammation, pulmonary histiocytosis X, pulmonary hypertension, surfactant deficiencies, pulmonary hypoplasia, pulmonary neoplasia, pulmonary nocardiosis, pulmonary tuberculosis, pulmonary veno-occlusive disease, rheumatoid lung disease, sarcoidosis, post-pneumonectomy, Wegener's granulomatosis, allergic granulomatosis, granulomatous vasculitides, eosinophilia, asthma and airway hyperreactivity (AHR), e.g., mild intermittent asthma, mild persistent asthma, moderate persistent asthma, severe persistent asthma, acute asthma, chronic asthma, atopic asthma, allergic asthma or idiosyncratic asthma, cystic fibrosis and associated conditions, e.g., allergic bronchopulmonary aspergillosis, chronic sinusitis, pancreatic insufficiency, lung or vascular inflammation, bacterial or viral infection, e.g., Haemophilus influenzae, S. aureus, Pseudomonas aeruginosa or RSV infection or an acute or chronic adult or pediatric respiratory distress syndrome (RDS) such as grade I, II, III or IV RDS or an RDS associated with, e.g., sepsis, pneumonia, reperfusion, atelectasis or chest trauma.

Chronic obstructive pulmonary diseases (COPDs) include conditions where airflow obstruction is located at upper airways, intermediate-sized airways, bronchioles or parenchyma, which can be manifested as, or associated with, tracheal stenosis, tracheal right ventricular hypertrophy pulmonary hypertension, polychondritis, bronchiectasis, bronchiolitis, e.g., idiopathic bronchiolitis, ciliary dyskinesia, asthma, emphysema, connective tissue disease, bronchiolitis of chronic bronchitis or lung transplantation.

Pulmonary fibrosis is a disease in which tissue deep in the lungs becomes thick and stiff, or scarred, over time. The formation of scar tissue is called fibrosis. As the lung tissue thickens, the lungs can't properly move oxygen into the bloodstream. As a result, the brain and other organs don't get the oxygen they need. Genetics may play a role in causing IPF. Pulmonary fibrosis where no known cause can be discerned is called idiopathic pulmonary fibrosis (IPF). IPF is a serious disease that usually affects middle-aged and older adults. IPF varies from person to person. in IPF, scarring begins in the air sac walls and the spaces around them. IPF has no cure yet. Many people live only about 3 to 5 years after diagnosis. The most common cause of death related to IPF is respiratory failure. Other causes of death include pulmonary hypertension, heart failure, pulmonary embolism, pneumonia, and lung cancer. Other names for IPF include: idiopathic diffuse interstitial pulmonary fibrosis, pulmonary fibrosis of unknown cause, pulmonary fibrosis, cryptogenic fibrosing alveolitis, usual interstitial pneumonitis and diffuse fibrosing alveolitis.

The methods described herein can also be used to treat or ameliorate acute or chronic asthma or their symptoms or complications, including airway epithelium injury, airway smooth muscle spasm or airway hyperresponsiveness, airway mucosa edema, increased mucus secretion, excessive T cell activation, or desquamation, atelectasis, corpulmonale, pneumothorax, subcutaneous emphysema, dyspnea, coughing, wheezing, shortness of breath, tachypnea, fatigue, decreased forced expiratory volume in the 1st second (FEV1), arterial hypoxemia, respiratory acidosis, inflammation including unwanted elevated levels of mediators such as IL-4, IL-5, IgE, histamine, substance P, neurokinin A, calcitonin gene-related peptide or arachidonic acid metabolites such as thromboxane or leukotrienes (LTD4 or LTC4), and cellular airway wall infiltration, e.g., by eosinophils, lymphocytes, macrophages or granulocytes.

Any of these lung diseases and disorders, and other respiratory or pulmonary conditions or symptoms are described elsewhere, e.g., The Merck Manual, 17.sup.th edition, M. H. Beers and R. Berkow editors, 1999, Merck Research Laboratories, Whitehouse Station, N.J., ISBN 0911910-10-7, or in other references cited herein it its entirety. In some of these conditions, where inflammation plays a role in the pathology of the condition, therapeutic agents used together with the non-mhLSCs can ameliorate or slow the progression of the condition by reducing damage from inflammation, such as damage to the lung epithelium. In other cases, therapeutic agents used together with the non-mhLSCs can act to limit pathogen replication or pathogen-associated lung tissue damage.

As used herein, the terms “administering,” “introducing”, “transplanting” and “implanting” are used interchangeably in the context of the placement of cells, e.g., non-mhLSCs, of the invention into a subject, by a method or route which results in at least partial localization of the introduced cells at a desired site, such as a site of injury or repair, such that a desired effect(s) is produced. The cells e.g., non-mhLSCs, or their differentiated progeny (e.g., respiratory epithelium-like cells) can be implanted directly to the respiratory airways, or alternatively be administered by any appropriate route which results in delivery to a desired location in the subject where at least a portion of the implanted cells or components of the cells remain viable. The period of viability of the cells after administration to a subject can be as short as a few hours, e.g., twenty-four hours, to a few days, to as long as several years, i.e., long-term engraftment. For example, in some embodiments of all aspects of the therapeutic methods described herein, an effective amount of an isolated or enriched population of isolated non-mhLSCs is administered directly to the lungs of an infant suffering from bronchopulmonary dysplasia by intratracheal administration. In other embodiments of all aspects of the therapeutic methods described herein, the population of isolated and enriched non-mhLSCs is administered via an indirect systemic route of administration, such as an intraperitoneal or intravenous route.

When provided prophylactically, the isolated and enriched non-mhLSCs can be administered to a subject in advance of any symptom of a respiratory disorder, e.g., asthma attack or for a cystic fibrosis subject. Accordingly, the prophylactic administration of an isolated or enriched for non-mhLSCs population serves to prevent a respiratory disorder, or further progress of respiratory diseases as disclosed herein.

When provided therapeutically, isolated and enriched non-mhLSCs are provided at (or after) the onset of a symptom or indication of a respiratory disorder, e.g., upon the onset of COPD.

As used herein, the terms “treat,” “treatment,” “treating,” or “amelioration” refer to therapeutic treatment, wherein the object is to reverse, alleviate, ameliorate, decrease, inhibit, or slow down the progression or severity of a condition associated with, a disease or disorder. The term “treating” includes reducing or alleviating at least one adverse effect or symptom of a condition, disease or disorder associated with an inflammatory disease, such as, but not limited to, asthma. Treatment is generally “effective” if one or more symptoms or clinical markers are reduced as that term is defined herein. Alternatively, treatment is “effective” if the progression of a disease is reduced or halted. That is, “treatment” includes not just the improvement of symptoms or markers, but also a cessation or at least slowing of progress or worsening of symptoms that would be expected in absence of treatment. Beneficial or desired clinical results include, but are not limited to, alleviation of one or more symptom(s), diminishment of extent of disease, stabilized (i.e., not worsening) state of disease, delay or slowing of disease progression, amelioration or palliation of the disease state, and remission (whether partial or total), whether detectable or undetectable. In some embodiments, “treatment” and “treating” can also mean prolonging survival of a subject as compared to expected survival if the subject did not receiving treatment.

As used herein, the term “prevention” refers to prophylactic or preventative measures wherein the object is to prevent or delay the onset of a disease or disorder, or delay the onset of symptoms of associated with a disease or disorder. In some embodiments, “prevention” refers to slowing down the progression or severity of a condition or the deterioration of lung function associated with a lung disease or disorder.

In another embodiment, “treatment” of a lung disease also includes providing relief from the symptoms or side-effects of the disease (including palliative treatment). For example, any reduction in inflammation, bronchospasm, bronchoconstriction, shortness of breath, wheezing, lower extremity edema, ascites, productive cough, hemoptysis, or cyanosis in a subject suffering from a respiratory disorder, such as asthma, no matter how slight, would be considered an alleviated symptom. In some embodiments of the aspects described herein, the symptoms or a measured parameter of a disease or disorder are alleviated by at least 5%, at least 10%, at least 20%, at least 30%, at least 40%, at least 50%, at least 60%, at least 70%, at least 80%, or at least 90%, upon administration of a population of isolated and enriched for LSCs, as compared to a control or non-treated subject.

Measured or measurable parameters include clinically detectable markers of disease, for example, elevated or depressed levels of a clinical or biological marker, as well as parameters related to a clinically accepted scale of symptoms or markers for a disease or disorder. It will be understood, however, that the total usage of the compositions as disclosed herein will be decided by the attending physician within the scope of sound medical judgment. The exact amount required will vary depending on factors such as the type of lung disease being treated, degree of damaged, whether the goal in for treatment or prevention or both, age of the subject, the amount of cells available etc. Thus, one of skill in the art realizes that a treatment may improve the disease condition, but may not be a complete cure for the disease.

In one embodiment of all aspects of the therapeutic methods described, the term “effective amount” as used herein refers to the amount of a population of isolated or enriched for non-mhLSCs needed to alleviate at least one or more symptoms of the respiratory disease or disorder, and relates to a sufficient amount of pharmacological composition to provide the desired effect, e.g., treat a subject having bronchopulmonary dysplasia. The term “therapeutically effective amount” therefore refers to an amount isolated and enriched for non-mhLSCs using the therapeutic methods as disclosed herein that is sufficient to effect a particular effect when administered to a typical subject, such as one who has or is at risk for bronchopulmonary dysplasia.

In another embodiment of all aspects of the methods described, an effective amount as used herein would also include an amount sufficient to prevent or delay the development of a symptom of the disease, alter the course of a symptom disease (for example but not limited to, slow the progression of a symptom of the disease), or even reverse a symptom of the disease. The effective amount of non-mhLSCs need for a particular effect will vary with each individual and will also vary with the type of lung disease addressed. Thus, it is not possible to specify the exact “effective amount”. However, for any given case, an appropriate “effective amount” can be determined by one of ordinary skill in the art using routine experimentation.

In some embodiments of all aspects of the therapeutic methods described, the subject is first diagnosed as having a disease or disorder affecting the lung tissue prior to administering the cells according to the methods described herein. In some embodiments of all aspects of the therapeutic methods described, the subject is first diagnosed as being at risk of developing lung disease or disorder prior to administering the cells, e.g., a long time smoker, a coal miner and a person having prior exposure to asbestos.

For use in all aspects of the therapeutic methods described herein, an effective amount of isolated non-mhLSCs comprises at least 10², at least 5×10², at least 10³, at least 5×10³ non-mhLSCs, at least 10⁴, at least 5×10⁴, at least 10⁵, at least 2×10⁵, at least 3×10⁵, at least 4×10⁵, at least 5×10⁵, at least 6×10⁵, at least 7×10⁵, at least 8×10⁵, at least 9×10⁵, or at least 1×10⁶ non-mhLSCs or multiples thereof per administration. In some embodiments, more than one administration of isolated non-mhLSCs is performed to a subject. The multiple administration of isolated non-mhLSCs can take place over a period of time. The non-mhLSCs can be isolated or enriched for from one or more donors, or can be obtained from an autologous source.

Exemplary modes of administration for use in the methods described herein include, but are not limited to, injection, intrapulmonary (including intranasal and intratracheal) infusion, inhalation (including intranasal), and ingestion. “injection” includes, without limitation, intravenous, intraarterial, intraventricular, intracardiac, transtracheal injection and infusion. The phrases “parenteral administration” and “administered parenterally” as used herein, refer to modes of administration other than enteral and topical administration, usually by injection, and includes, without limitation, intravenous, intraventricular, intracardiac, transtracheal injection and infusion.

In preferred embodiments of all aspects of the therapeutic methods described, an effective amount of isolated non-mhLSCs is administered to a subject by intrapulmonary administration or delivery. As defined herein, intrapulmonary′ administration or intrapulmonary delivery refers to all routes of administration whereby a population of isolated and enriched for non-mhLSCs, is administered in a way that results in direct contact of these cells with the airways of a subject, including, but not limited to, transtracheal, intratracheal, and intranasal administration. In such embodiments, the cells are injected into the nasal passages or trachea. In some embodiments, the cells are directly inhaled by a subject. In some embodiments of all aspects of the therapeutic methods described, intrapulmonary delivery of cells includes administration methods whereby cells are administered, for example as a cell suspension, to an intubated subject via a tube placed in the trachea or “tracheal intubation.”

As used herein, “tracheal intubation” refers to the placement of a flexible tube, such as a plastic tube, into the trachea. The most common tracheal intubation, termed herein as “orotracheal intubation” is where, with the assistance of a laryngoscope, an endotracheal tube is passed through the mouth, larynx, and vocal cords, into the trachea. A bulb is then inflated near the distal tip of the tube to help secure it in place and protect the airway from blood, vomit, and secretions. In some embodiments of all aspects of the therapeutic methods described, cells are administered to a subject having “nasotracheal intubation,” which is defined as a tracheal intubation where a tube is passed through the nose, larynx, vocal cords, and trachea.

In some embodiments of all aspects of the therapeutic methods described, an effective amount of isolated and enriched non-mhLSCs is administered to a subject by systemic administration, such as intravenous administration.

The phrases “systemic administration,” “administered systemically”, “peripheral administration” and “administered peripherally” as used herein refer to the administration of population of non-mhLSCs other than directly into the lung, such that it enters, instead, the subject's circulatory system.

In some embodiments of all aspects of the therapeutic methods described, one or more routes of administration are used in a subject to achieve distinct effects. For example, isolated or enriched population of non-mhLSCs are administered to a subject by both intratracheal and intraperitoneal administration routes for treating or repairing respiratory epithelium and for pulmonary vascular repair and regeneration respectively. In such embodiments, different effective amounts of the isolated or enriched non-mhLSCs can be used for each administration route.

In some embodiments of all aspects of the therapeutic methods described, the methods further comprise administration of one or more therapeutic agents, such as a drug or a molecule, that can enhance or potentiate the effects mediated by the administration of the isolated or enriched non-mhLSCs, such as enhancing homing or engraftment of the non-mhLSCs, increasing repair of respiratory epithelia, or increasing growth and regeneration of pulmonary vasculature, i.e., vascular regeneration. The therapeutic agent can be a protein (such as an antibody or antigen-binding fragment), a peptide, a polynucleotide, an aptamer, a virus, a small molecule, a chemical compound, a cell, a drug, etc. As defined herein, “vascular regeneration” refers to de novo formation of new blood vessels or the replacement of damaged blood vessels (e.g., capillaries) after injuries or traumas, as described herein, including but not limited to, respiratory disease. “Angiogenesis” is a term that can be used interchangeably to describe such phenomena.

In some embodiments of all aspects of the therapeutic methods described, the methods further comprise administration of one or more together with growth, differentiation, and angiogenesis agent or factor that are known in the art to stimulated cell growth, differentiation, and angiogenesis in the lung tissue. In some embodiments, any one of these factors can be delivered to prior to or after administering the compositions described herein. Multiple subsequent delivery of any one of these factors can also occur to induce and/or enhance the engraftment, differentiation and/or angiogenesis. Suitable growth factors include but are not limited to transforming growth factor-beta (TGFβ), vascular endothelial growth factor (VEGF), platelet derived growth factor (PDGF), angiopoietins, epidermal growth factor (EGF), bone morphogenic protein (BMP), basic fibroblast growth factor (bFGF), insulin and 3-isobutyl-1-methylxasthine (IBMX). Other examples are described in Dijke et al., “Growth Factors for Wound Healing”, Bio/Technology, 7:793-798 (1989); Mulder G D, Haberer P A, Jeter K F, eds. Clinicians' Pocket Guide to Chronic Wound Repair. 4th ed. Springhouse, Pa.: Springhouse Corporation; 1998:85; Ziegler T. R., Pierce, G. F., and Herndon, D. N., 1997, international Symposium on Growth Factors and Wound Healing: Basic Science & Potential Clinical Applications (Boston, 1995, Serono Symposia USA), Publisher: Springer Verlag, and these are hereby incorporated by reference in their entirety.

In one embodiment, the composition can include one or more bioactive agents to induce healing or regeneration of damaged tissue, such as recruiting blood vessel forming cells from the surrounding tissues to provide connection points for the nascent vessels. Suitable bioactive agents include, but are not limited to, pharmaceutically active compounds, hormones, growth factors, enzymes, DNA, RNA, siRNA, viruses, proteins, lipids, polymers, hyaluronic acid, pro-inflammatory molecules, antibodies, antibiotics, anti-inflammatory agents, anti-sense nucleotides and transforming nucleic acids or combinations thereof. Other bioactive agents can promote increase mitosis for cell growth and cell differentiation.

A great number of growth factors and differentiation factors that are known in the art to stimulated cell growth and differentiation of the stem cells and progenitor cells. Suitable growth factors and cytokines include any cytokines or growth factors capable of stimulating, maintaining, and/or mobilizing progenitor cells. They include but are not limited to stem cell factor (SCF), granulocyte-colony stimulating factor (G-CSF), granulocyte-macrophage stimulating factor (GM-CSF), stromal cell-derived factor-1, steel factor, vascular endothelial growth factor (VEGF), TGFIβ, platelet derived growth factor (PDGF), angiopoeitins (Ang), epidermal growth factor (EGF), bone morphogenic protein (BMP), fibroblast growth factor (FGF), hepatocye growth factor, insulin-like growth factor (IGF-1), interleukin (IL)-3, IL-1α, IL-1β), IL-6, IL-7, IL-8, IL-11, and IL-13, colony-stimulating factors, thrombopoietin, erythropoietin, fit3-ligand, and tumor necrosis factor α. Other examples are described in Dijke et al., “Growth Factors for Wound Healing”, Bio/Technology, 7:793-798 (1989); Mulder G D, Haberer P A, Jeter K F, eds. Clinicians' Pocket Guide to Chronic Wound Repair. 4th ed. Springhouse, Pa.: Springhouse Corporation; 1998:85; Ziegler T. R., Pierce, G. F., and Herndon, D. N., 1997, International Symposium on Growth Factors and Wound Healing: Basic Science & Potential Clinical Applications (Boston, 1995, Serono Symposia USA), Publisher: Springer Verlag.

In one embodiment of all aspects of the therapeutic methods described, the composition described is a suspension of non-mhLSCs in a suitable physiologic carrier solution such as saline. The suspension can contain additional bioactive agents include, but are not limited to, pharmaceutically active compounds, hormones, growth factors, enzymes, DNA, RNA, siRNA, viruses, proteins, lipids, polymers, hyaluronic acid, pro-inflammatory molecules, antibodies, antibiotics, anti-inflammatory agents, anti-sense nucleotides and transforming nucleic acids or combinations thereof.

In certain embodiments of all aspects of the therapeutic methods described, the therapeutic agent is a “pro-angiogenic factor,” which refers to factors that directly or indirectly promote new blood vessel formation. The pro-angiogenic factors include, but are not limited to epidermal growth factor (EGF), E-cadherin, VEGF, angiogenin, angiopoietin-1, fibroblast growth factors: acidic (aFGF) and basic (bFGF), fibrinogen, fibronectin, heparanase, hepatocyte growth factor (HGF), angiopoietin, hypoxia-inducible factor-1 (HIF-1), insulin-like growth factor-1 (IGF-1), IGF, BP-3, platelet-derived growth factor (PDGF), VEGF-A, VEGF-C, pigment epithelium-derived factor (PEDF), vascular permeability factor (VPF), vitronection, leptin, trefoil peptides (TFFs), CYR61 (CCN1), NOV (CCN3), leptin, midkine, placental growth factor platelet-derived endothelial cell growth factor (PD-ECGF), platelet-derived growth factor-BB (PDGF-BB), pleiotrophin (PTN), progranulin, proliferin, transforming growth factor-alpha (TGF-alpha), transforming growth factor-beta (TGF-beta), tumor necrosis factor-alpha (TNF-alpha), c-Myc, granulocyte colony-stimulating factor (G-CSF), stromal derived factor 1 (SDF-1), scatter factor (SF), osteopontin, stem cell factor (SCF), matrix metalloproteinases (MMPs), thrombospondin-1 (TSP-1), pleitrophin, proliferin, follistatin, placental growth factor (PIGF), midkine, platelet-derived growth factor-BB (PDGF), and fractalkine, and inflammatory cytokines and chemokines that are inducers of angiogenesis and increased vascularity, e.g., interleukin-3 (IL-3), interleukin-8 (IL-8), CCL2 (MCP-1), interleukin-8 (IL-8) and CCL5 (RANTES). Suitable dosage of one or more therapeutic agents can include a concentration of about 0.1 to about 500 ng/ml, about 10 to about 500 ng/ml, about 20 to about 500 ng/ml, about 30 to about 500 ng/ml, about 50 to about 500 ng/ml, or about 80 ng/ml to about 500 ng/ml. In some embodiments, the suitable dosage of one or more therapeutic agents is about 10, about 25, about 45, about 60, about 75, about 100, about 125, about 150, about 175, about 200, about 225, about 250, about 275, about 300, about 325, about 350, about 375, about 400, about 425, about 450, about 475, or about 500 ng/ml. In other embodiments, suitable dosage of one or more therapeutic agents is about 0.6, about 0.7, about 0.8, about 0.9, about 1.0, about 1.5, or about 2.0 μg/ml.

In some embodiments of all aspects of the therapeutic methods described, the methods further comprise administration of one or more surfactants as therapeutic agents, or may be used in combination with one or more surfactant therapies. Surfactant, as used herein, refers to any surface active agent, including but not limited to wetting agents, surface tension depressants, detergents, dispersing agents and emulsifiers. Particularly preferred are those that from a monomolecular layer over pulmonary alveolar surfaces, including but not limited to lipoproteins, lecithins, phosphatidylglycerol (PG), dipalmitoyl-phosphatidyl choline (DPPG), apoprotein A, apoprotein B, apoprotein C, apoprotein D, palmitoyl oleoyl, phosphatidyl glycerol palmitic and sphygomyelins. Exemplary surfactants include, but are not limited to surfactant protein A, surfactant protein B, surfactant protein C, surfactant protein D, and mixtures and combinations thereof. Commercially available surfactants include, but are not limited to, KL-4, SURVANTA®, bovine lipid extract surfactant (BLES), INFASURF® (CALFACTANT®), CUROSURF®, HL-10, AEROSURF®, SUBOXONE®. ALVEOFACT®, SURFAXIN®, VENTICUTE®, PUMACTANT®/ALEC, and EXOSURF®.

In some embodiments of all aspects of the therapeutic methods described. administration of one or more other standard therapeutic agents can be combined with the administration of the enriched non-mhLSCs to treat the respiratory disorders or conditions, e.g., asthma, RDS or COPD, including the use of anticholinergic agents, β-2-adrenoreceptor agonists, such as formoterol or salmeterol, corticosteroids, antibiotics, anti-oxidation, antihypertension agents, nitric oxide, caffeine, dexamethasome, and IL-10 or other cytokines. In some embodiments, the included standard therapeutic agents are used for treating the symptoms of the lung disease. Table 1 shows some of the standard medical therapy for the some lung diseases.

For example, the use of non-mhLSCs in the methods described herein to treat, ameliorate or slow the progression of a condition such as CF can be optionally combined with other suitable treatments or therapeutic agents. For CF, this includes, but is not limited to, oral or aerosol corticosteroid treatment, ibuprofen treatment, DNAse or IL-10 treatment, diet control, e.g., vitamin E supplementation, vaccination against pathogens, e.g., Haemophilus influenzae, chest physical therapy, e.g., chest drainage or percussion, or any combination therein.

In some embodiments of all aspects of the therapeutic methods described, the standard therapeutic agents are those that have been described in detail, see, e.g., Harrison's Principles of Internal Medicine, 15.sup.th edition, 2001, E. Braunwald, et al., editors, McGraw-Hill, New York, N.Y., ISBN 0-07-007272-8, especially chapters 252-265 at pages 1456-1526; Physicians Desk Reference 54.sup.th edition. 2000, pages 303-3251, ISBN 1-56363-330-2, Medical Economics Co., Inc., Montvale, N.J. Treatment of any of lung disease, respiratory or pulmonary condition can be accomplished using the treatment regimens described herein. For chronic conditions, intermittent dosing can be used to reduce the frequency of treatment. Intermittent dosing protocols are as described herein.

For the clinical use of the methods described herein, isolated or enriched populations of enriched non-mhLSCs described herein can be administered along with any pharmaceutically acceptable compound, material, carrier or composition which results in an effective treatment in the subject. Thus, a pharmaceutical formulation for use in the methods described herein can contain an isolated or enriched population of non-mhLSCs in combination with one or more pharmaceutically acceptable ingredients.

The term “carrier” refers to a diluent, adjuvant, excipient, or vehicle with which the therapeutic is administered. Such pharmaceutical carriers can be sterile liquids, such as water and oils, including those of petroleum, animal, vegetable or synthetic origin, such as peanut oil, soybean oil, mineral oil, sesame oil and the like. Water is a preferred carrier when the pharmaceutical composition is administered intravenously. Saline solutions and aqueous dextrose and glycerol solutions can also be employed as liquid carriers, particularly for injectable solutions. Suitable pharmaceutical excipients include starch. glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. The composition, if desired, can also contain minor amounts of wetting or emulsifying agents, or pH buffering agents. These compositions can take the form of solutions, suspensions, emulsion, tablets, pills, capsules, powders, sustained-release formulations, and the like. The composition can be formulated as a suppository, with traditional binders and carriers such as triglycerides. Oral formulation can include standard carriers such as pharmaceutical grades of mannitol, lactose, starch, magnesium stearate, sodium saccharine, cellulose, magnesium carbonate, etc. Examples of suitable pharmaceutical carriers are described in Remington's Pharmaceutical Sciences, 18th Ed., Gennaro, ed. (Mack Publishing Co., 1990). The formulation should suit the mode of administration.

In one embodiment, the term “pharmaceutically acceptable” means approved by a regulatory agency of the Federal or a state government or listed in the U.S. Pharmacopeia or other generally recognized pharmacopeia for use in animals, and more particularly in humans. Specifically, it refers to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The phrase “pharmaceutically acceptable carrier” as used herein means a pharmaceutically acceptable material, composition or vehicle, such as a liquid or solid filler, diluent, excipient, solvent, media (e.g., stem cell media), encapsulating material, manufacturing aid (e.g., lubricant, talc magnesium, calcium or zinc stearate, or steric acid), or solvent encapsulating material, involved in maintaining the activity of, carrying, or transporting the isolated or enriched populations of LSCs from one organ, or portion of the body, to another organ, or portion of the body.

Each carrier must be “acceptable” in the sense of being compatible with the other ingredients of the formulation and not injurious to the patient. Some examples of materials which can serve as pharmaceutically-acceptable carriers include: (1) sugars, such as lactose, glucose and sucrose; (2) phosphate buffered solutions; (3) pyrogen-free water; (4) isotonic saline; (5) malt; (6) gelatin; (7) lubricating agents, such as magnesium stearate, sodium lauryl sulfate and talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol (PEG); (12) esters, such as ethyl oleate and ethyl laurate; (13) agar, (14) buffering agents, such as magnesium hydroxide and aluminum hydroxide; (15) alginic acid; (16) cellulose, and its derivatives, such as sodium carboxymethyl cellulose, methylcellulose, ethyl cellulose, microcrystalline cellulose and cellulose acetate; (17) powdered tragacanth; (18) Ringer's solution; (19) ethyl alcohol; (20) pH buffered solutions; (21) polyesters, polycarbonates and/or polyanhydrides; (22) bulking agents, such as polypeptides and amino acids (23) serum component, such as serum albumin, HDL and LDL; (24) C2-C12 alcohols, such as ethanol; (25) starches, such as corn starch and potato starch; and (26) other non-toxic compatible substances employed in pharmaceutical formulations. Wetting agents, coloring agents, release agents, coating agents, sweetening agents, flavoring agents, perfuming agents, preservative and antioxidants can also be present in the formulation. The terms such as “excipient”, “carrier”, “pharmaceutically acceptable carrier” or the like are used interchangeably herein.

### Definitions

Unless otherwise defined, all technical and scientific terms used herein have the same meaning as commonly understood by one of ordinary skill in the art to which this invention belongs. Certain terms employed herein, in the specification, examples and claims are collected here.

As used herein, in vivo (Latin for “within the living”) refers to those methods using a whole, living organism, such as a human subject. As used herein, “ex vivo” (Latin: out of the living) refers to those methods that are performed outside the body of a subject, and refers to those procedures in which an organ, cells, or tissue are taken from a living subject for a procedure, e.g., isolating non-mhLSCs from a lung tissue obtained from a donor subject, and then administering the isolated non-mhLSCs sample to a recipient subject. As used herein, “in vitro” refers to those methods performed outside of a subject, such as an in vitro cell culture experiment. For example, isolated non-mhLSCs can be cultured in vitro to expand or increase the number of non-mhLSCs, or to direct differentiation of the non-mhLSCs to a specific lineage or cell type, e.g., respiratory epithelial cells, prior to being used or administered according to the methods described herein.

The term “pluripotent” as used herein refers to a cell with the capacity, under different conditions, to commit to one or more specific cell type lineage and differentiate to more than one differentiated cell type of the committed lineage, and preferably to differentiate to cell types characteristic of all three germ cell layers. Pluripotent cells are characterized primarily by their ability to differentiate to more than one cell type, preferably to all three germ layers, using, for example, a nude mouse teratoma formation assay. Pluripotency is also evidenced by the expression of embryonic stem (ES) cell markers, although the preferred test for pluripotency is the demonstration of the capacity to differentiate into cells of each of the three germ layers. It should be noted that simply culturing such cells does not, on its own, render them pluripotent. Reprogrammed pluripotent cells (e.g., iPS cells as that term is defined herein) also have the characteristic of the capacity of extended passaging without loss of growth potential, relative to primary cell parents, which generally have capacity for only a limited number of divisions in culture.

The term “progenitor” cell are used herein refers to cells that have a cellular phenotype that is more primitive (i.e., is at an earlier step along a developmental pathway or progression than is a fully differentiated or terminally differentiated cell) relative to a cell which it can give rise to by differentiation. Often, progenitor cells also have significant or very high proliferative potential. Progenitor cells can give rise to multiple distinct differentiated cell types or to a single differentiated cell type, depending on the developmental pathway and on the environment in which the cells develop and differentiate. Progenitor cells give rise to precursor cells of specific determine lineage, for example, certain lung progenitor cells divide to give pulmonary epithelial lineage precursor cells. These precursor cells divide and give rise to many cells that terminally differentiate to pulmonary epithelial cells.

The term “precursor” cell are used herein refers to cells that have a cellular phenotype that is more primitive than a terminally differentiated cell but is less primitive than a stem cell or progenitor cells that is along its same developmental pathway. A “precursor” cell is typically progeny cells of a “progenitor” cell which are some of the daughter of “stem cells”. One of the daughters in a typical asymmetrical cell division assumes the role of the stem cell.

The term “embryonic stem cell” is used to refer to the pluripotent stem cells of the inner cell mass of the embryonic blastocyst (see U.S. Pat. Nos. 5,843,780, 6,200,806). Such cells can similarly be obtained from the inner cell mass of blastocysts derived from somatic cell nuclear transfer (see, for example, U.S. Pat. Nos. 5,945,577, 5,994,619, 6,235,970). The distinguishing characteristics of an embryonic stem cell define an embryonic stem cell phenotype. Accordingly, a cell has the phenotype of an embryonic stem cell if it possesses one or more of the unique characteristics of an embryonic stem cell such that that cell can be distinguished from other cells. Exemplary distinguishing embryonic stem cell characteristics include, without limitation, gene expression profile, proliferative capacity, differentiation capacity, karyotype, responsiveness to particular culture conditions, and the like.

The term “adult stem cell” is used to refer to any multipotent stem cell derived from non-embryonic tissue, including fetal, juvenile, and adult tissue. In some embodiments, adult stem cells can be of non-fetal origin. Stem cells have been isolated from a wide variety of adult tissues including blood, bone marrow, brain, olfactory epithelium, skin, pancreas, skeletal muscle, and cardiac muscle. Each of these stem cells can be characterized based on gene expression, factor responsiveness, and morphology in culture. Exemplary adult stem cells include neural stem cells, neural crest stem cells, mesenchymal stem cells, hematopoietic stem cells, and pancreatic stem cells. As indicated above, stem cells have been found resident in virtually every tissue. Accordingly, the present invention appreciates that stem cell populations can be isolated from virtually any animal tissue.

In the context of cell ontogeny, the adjective “differentiated”, or “differentiating” is a relative term meaning a “differentiated cell” is a cell that has progressed further down the developmental pathway than the cell it is being compared with. Thus, stem cells can differentiate to lineage-restricted precursor cells (such as a lung stem cell), which in turn can differentiate into other types of precursor cells further down the pathway (such as a thymocyte, or a T lymphocyte precursor), and then to an end-stage differentiated cell, which plays a characteristic role in a certain tissue type, and may or may not retain the capacity to proliferate further.

The term “differentiated cell” is meant any primary cell that is not, in its native form, pluripotent as that term is defined herein. Stated another way, the term “differentiated cell” refers to a cell of a more specialized cell type derived from a cell of a less specialized cell type (e.g., a stem cell such as a lung stem cell) in a cellular differentiation process. Without wishing to be limited to theory, a pluripotent stem cell in the course of normal ontogeny can differentiate first to an endothelial cell that is capable of forming hematopoietic stem cells and other cell types. Further differentiation of a lung stem cell leads to the formation of the various pulmonary cell types, including pneumocyte type I and II cell types, endothelial cell types, smooth muscle, and epithelial cells.

As used herein, the term “somatic cell” refers to are any cells forming the body of an organism, as opposed to germline cells. In mammals, germline cells (also known as “gametes”) are the spermatozoa and ova which fuse during fertilization to produce a cell called a zygote, from which the entire mammalian embryo develops. Every other cell type in the mammalian body—apart from the sperm and ova, the cells from which they are made (gametocytes) and undifferentiated stem cells—is a somatic cell: internal organs, skin, bones, blood, and connective tissue are all made up of somatic cells. In some embodiments the somatic cell is a “non-embryonic somatic cell”, by which is meant a somatic cell that is not present in or obtained from an embryo and does not result from proliferation of such a cell in vitro. In some embodiments the somatic cell is an “adult somatic cell”, by which is meant a cell that is present in or obtained from an organism other than an embryo or a fetus or results from proliferation of such a cell in vitro.

As used herein, the term “adult cell” refers to a cell found throughout the body after embryonic development.

The term “phenotype” refers to one or a number of total biological characteristics that define the cell or organism under a particular set of environmental conditions and factors, regardless of the actual genotype. For example, the expression of cell surface markers in a cell.

The term “cell culture medium” (also referred to herein as a “culture medium” or “medium”) as referred to herein is a medium for culturing cells containing nutrients that maintain cell viability and support proliferation. The cell culture medium may contain any of the following in an appropriate combination: salt(s), buffer(s), amino acids, glucose or other sugar(s), antibiotics, serum or serum replacement, and other components such as peptide growth factors, etc. Cell culture media ordinarily used for particular cell types are known to those skilled in the art.

The terms “renewal” or “self-renewal” or “proliferation” are used interchangeably herein, are used to refer to the ability of stem cells to renew themselves by dividing into the same non-specialized cell type over long periods, and/or many months to years.

In some instances, “proliferation” refers to the expansion of cells by the repeated division of single cells into two identical daughter cells.

The term “lineages” is used herein describes a cell with a common ancestry or cells with a common developmental fate.

The term “isolated cell” as used herein refers to a cell that has been removed from an organism in which it was originally found or a descendant of such a cell. Optionally the cell has been cultured in vitro, e.g., in the presence of other cells. Optionally the cell is later introduced into a second organism or re-introduced into the organism from which it (or the cell from which it is descended) was isolated.

The term “isolated population” with respect to an isolated population of cells as used herein refers to a population of cells that has been removed and separated from a mixed or heterogeneous population of cells. In some embodiments, an isolated population is a substantially pure population of cells as compared to the heterogeneous population from which the cells were isolated or enriched from.

The term “tissue” refers to a group or layer of specialized cells which together perform certain special functions. The term “tissue-specific” refers to a source of cells from a specific tissue.

The terms “decrease”, “reduced”, “reduction”, “decrease” or “inhibit” are all used herein generally to mean a decrease by a statistically significant amount. However, for avoidance of doubt, “reduced”, “reduction” or “decrease” or “inhibit” typically means a decrease by at least about 5%-10% as compared to a reference level, for example a decrease by at least about 20%, or at least about 30%, or at least about 40%, or at least about 50%, or at least about 60%, or at least about 70%, or at least about 80%, or at least about 90% decrease (i.e., absent level as compared to a reference sample), or any decrease between 10-90% as compared to a reference level. In the context of treatment or prevention, the reference level is a symptom level of a subject in the absence of administering a population of non-mhLSCs.

The terms “increased”, “increase” or “enhance” are all used herein to generally mean an increase by a statically significant amount; for the avoidance of any doubt, the terms “increased”, “increase” or “enhance” means an increase of at least 10% as compared to a reference level, for example an increase of at least about 20%, or at least about 30%, or at least about 40%, or at least about 50%, or at least about 60%, or at least about 70%, or at least about 80%, or at least about 90% increase or more or any increase between 10-90% as compared to a reference level, or at least about a 2-fold, or at least about a 3-fold, or at least about a 4-fold, or at least about a 5-fold or at least about a 10-fold increase, or any increase between 2-fold and 10-fold or greater as compared to a reference level. In the context of non-mhLSCs expansion in vitro, the reference level is the initial number of non-mhLSCs isolated from the lung tissue sample.

The term “statistically significant” or “significantly” refers to statistical significance and generally means a two standard deviation (2SD) below normal, or lower, concentration of the marker. The term refers to statistical evidence that there is a difference. It is defined as the probability of making a decision to reject the null hypothesis when the null hypothesis is actually true. The decision is often made using the p-value.

As used herein the term “comprising” or “comprises” is used in reference to compositions, methods, and respective component(s) thereof, that are essential to the invention, yet open to the inclusion of unspecified elements, whether essential or not.

The term “consisting of” refers to compositions, methods, and respective components thereof as described herein, which are exclusive of any element not recited in that description of the embodiment.

Unless otherwise explained, all technical and scientific terms used herein have the same meaning as commonly understood by one of ordinary skill in the art to which this disclosure belongs. Definitions of common terms in molecular biology may be found in Benjamin Lewin, Genes IX, published by Jones & Bartlett Publishing, 2007 (ISBN-13: 9780763740634); Kendrew et al. (eds.), The Encyclopedia of Molecular Biology, published by Blackwell Science Ltd., 1994 (ISBN 0-632-02182-9); and Robert A. Meyers (ed.), Molecular Biology and Biotechnology: a Comprehensive Desk Reference, published by VCH Publishers, Inc., 1995 (ISBN 1-56081-569-8). Further, unless otherwise required by context, singular terms shall include pluralities and plural terms shall include the singular.

Unless otherwise stated, the present invention was performed using standard procedures known to one skilled in the art, for example, in Maniatis et al., Molecular Cloning: A Laboratory Manual, Cold Spring Harbor Laboratory Press, Cold Spring Harbor, N.Y., USA (1982); Sambrook et al., Molecular Cloning: A Laboratory Manual (2 ed.), Cold Spring Harbor Laboratory Press, Cold Spring Harbor, N.Y., USA (1989); Davis et al., Basic Methods in Molecular Biology, Elsevier Science Publishing, Inc., New York, USA (1986); Current Protocols in Molecular Biology (CPMB) (Fred M. Ausubel, et al. ed., John Wiley and Sons, Inc.), Current Protocols in Immunology (CPI) (John E. Coligan, et. al., ed. John Wiley and Sons, Inc.), Current Protocols in Cell Biology (CPCB) (Juan S. Bonifacino et. al. ed., John Wiley and Sons, Inc.), Culture of Animal Cells: A Manual of Basic Technique by R. Ian Freshney, Publisher: Wiley-Liss; 5th edition (2005) and Animal Cell Culture Methods (Methods in Cell Biology, Vol. 57, Jennie P. Mather and David Barnes editors, Academic Press, 1st edition, 1998) which are all herein incorporated by reference in their entireties.

It should be understood that this invention is not limited to the particular methodology, protocols, and reagents, etc., described herein and as such may vary. The terminology used herein is for the purpose of describing particular embodiments only, and is not intended to limit the scope of the present invention, which is defined solely by the claims.

Other than in the operating examples, or where otherwise indicated, all numbers expressing quantities of ingredients or reaction conditions used herein should be understood as modified in all instances by the term “about.” The term “about” when used in connection with percentages will mean±1%.

All patents and publications identified are expressly incorporated herein by reference for the purpose of describing and disclosing, for example, the methodologies described in such publications that might be used in connection with the present invention. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents is based on the information available to the applicants and does not constitute any admission as to the correctness of the dates or contents of these documents.

In some embodiments, the present invention can be defined in any of the following alphabetized paragraphs:
[A] A pharmaceutical composition comprising: an enriched population of isolated c-kit positive lung stem cells from a human lung tissue sample wherein the c-kit positive lung stem cells are negative for the CD44, CD73 and CD105 markers of the mesenchymal stromal cell lineage (non-mhLSCs); and a pharmaceutically acceptable carrier.
[B] The pharmaceutical composition of paragraph [A], wherein the lung tissue is from an adult.
[C] The pharmaceutical composition of paragraph [A] or [B], wherein the non-mhLSCs are further expanded ex vivo.
[D] A method of preparing an isolated population of lung stem cells positive for c-kit and negative for the CD44, CD73 and CD105 markers of the mesenchymal stromal cell lineage (non-mhLSCs), wherein the non-mhLSCs are in a pool of c-kit-positive human lung stem cells (hLSCs) comprised of non-mhLSCs and mesenchymal-like lung stem cells that are positive for c-kit and the CD44, CD73 and CD105 markers (ml-hLSCs), the method comprising: obtaining human lung tissue from a subject; selecting non-mhLSCs from the pool of hLSCs from the human lung tissue; and proliferating said cells in a culture medium.
[E] A method of proliferating an isolated population of lung stem cells positive for c-kit and negative for the CD44, CD73 and CD105 markers of the mesenchymal stromal cell lineage (non-mhLSCs), wherein the non-mhLSCs are in a pool of c-kit-positive human lung stem cells (hLSCs) comprised of non-mhLSCs and mesenchymal-like lung stem cells that are positive for c-kit and the CD44, CD73 and CD105 markers (ml-hLSCs), the method comprising: selecting at least one non-mhLSC from the pool of hLSCs from a human lung tissue sample; introducing said at least one selected non-mhLSC to a culture medium; and proliferating said at least one selected non-mhLSC in the culture medium.
[F] A method for treating or preventing a lung disease or disorder in a subject in need thereof, comprising: obtaining a human lung tissue from the subject in need thereof or from a different subject; extracting a population of stem cells positive for c-kit and negative for the CD44, CD73 and CD105 markers of the mesenchymal stromal cell lineage (non-mhLSCs) from said lung tissue; expanding said population of non-mhLSCs; and administering said expanded population of non-mhLSCs to the subject in need thereof.
[G] A method of repairing and/or regenerating damaged lung tissue in a subject in need thereof comprising: extracting a population of stem cells positive for c-kit and negative for the CD44, CD73 and CD105 markers of the mesenchymal stromal cell lineage (non-mhLSCs) from lung tissue; culturing and expanding said population of non-mhLSCs; and administering a dose of said extracted and expanded population of non-mhLSCs to an area of damaged lung tissue in the subject effective to repair and/or regenerate the damaged lung tissue.
[H] The method of any one of paragraphs [D]-[G], wherein the human lung tissue is an adult lung tissue.

The method of any one of paragraphs [D]-[H], wherein the human lung tissue is cryopreserved prior to selecting or extracting non-mhLSCs.
[J] The method of any one of paragraphs [D]-[I], wherein the selecting or extracting of non-mhLSCs is performed using an antibody against c-kit.
[K] The method of any of one paragraphs [D]-[J] further comprising negative selection for the CD44, CD73 and CD105 markers of the mesenchymal stromal cell lineage.
[L] The method of any paragraphs [D]-[K], wherein the selecting is by flow cytometry.
[M] The method of any paragraphs [D]-[K], wherein the selecting is by immunomagnetic selection with c-kit antibodies conjugated to beads.
[N] The method of any of one of paragraphs [D]-[M], further comprising cryopreserving the non-mhLSCs.
[O] The method of any one of paragraphs [F]-[N], further comprising administering at least one therapeutic agent.
[P] The method of any one of paragraphs [[F]-[O], wherein the population of non-mhLSCs repairs, reconstitutes and/or generates pulmonary epithelium, pulmonary vasculature/pulmonary endothelium and/or pulmonary alveoli.
[Q] The method of any one of paragraphs [F]-[P], further comprising selecting a subject who is suffering from a lung disease or disorder prior to administering the population enriched for non-mhLSCs.
[R] The method of any one of paragraphs [F]-[Q], further comprising selecting a subject in need of restoring the structural and functional integrity of a damaged lung prior to administering the non-mhLSCs.
[S] The method of any one of paragraphs [F]-[R], further comprising selecting a subject in need of treatment, prevention, repair, reconstitution or generation of pulmonary vasculature or pulmonary epithelium, pulmonary endothelium, or pulmonary alveoli prior to administering the cells.
[T] The method of any one of paragraphs [F]-[S], wherein the administration is intrapulmonary administration, systemic administration, intravenous administration, or a combination thereof
[U] The method of paragraph [T], wherein the intrapulmonary administration is intratracheal or intranasal administration.
[V] A composition for use in treating and/or preventing a lung disease or disorder in a subject, the composition comprising an enriched population of isolated c-kit positive lung stem cells from a human lung tissue sample wherein the c-kit positive lung stem cells are negative for the CD44, CD73 and CD105 markers of the mesenchymal stromal cell lineage (non-mhLSCs).
[W] The composition of paragraph [V], wherein the lung tissue is from an adult.
[X] The composition of paragraph [V] or [W], wherein the c-kit cells are further expanded ex vivo.
[Y] A method for treating or preventing a lung disorder in a subject in need thereof, comprising administering a pharmaceutical composition of any one of paragraphs [A]-[C].
[Z] A method for treating or preventing a lung disorder in a subject in need thereof, comprising administering a composition of any one of paragraphs [V]-[X].
[AA] The method of paragraph [Y] or [Z], further comprising administering at least one therapeutic agent.
[BB] The method of any one of paragraphs [Y]-[AA], wherein the population of non-mhLSCs repairs, reconstitutes and/or generates pulmonary epithelium, pulmonary vasculature/pulmonary endothelium and/or pulmonary alveoli.
[CC] The method of any one of paragraphs [Y]-[BB] further comprising selecting a subject who is suffering from a lung disorder prior to administering the population enriched for non-mhLSCs.
[DD] The method of any one of paragraphs [Y]-[CC] further comprising selecting a subject in need of restoring the structural and functional integrity of a damaged lung prior to administering the cells.
[EE] The method of any one of paragraphs [Y]-[DD] further comprising selecting a subject in need of treatment, prevention, repair, reconstitution or generation of pulmonary vasculature or pulmonary epithelium, pulmonary endothelium, or pulmonary alveoli prior to administering the cells.
[FF] The method of any one of paragraphs [Y]-[EE], wherein the administration is intrapulmonary administration, systemic administration, intravenous administration, or a combination thereof
[GG] The method of paragraph [FF], wherein the intrapulmonary administration is intratracheal or intranasal administration.

This invention is further illustrated by the following example which should not be construed as limiting. The contents of all references cited throughout this application, as well as the figures and table are incorporated herein by reference.

Those skilled in the art will recognize, or be able to ascertain using not more than routine experimentation, many equivalents to the specific embodiments of the invention described herein, different culture medium and supplements can be used to culture expand the isolated cells. One skilled in the art would be able to perform tests to evaluate the choice of culture medium and supplements. Such equivalents are intended to be encompassed by the following claims.

The references cited herein and throughout the specification are incorporated herein by reference.

### Example

Chronic obstructive pulmonary disease (COPD) in humans is characterized by chronic inflammation, enlargement of bronchioles and alveoli, destruction of the alveolar walls, fibrosis and, ultimately, respiratory failure (1-6). Telomere attrition and cellular senescence enhance the susceptibility to emphysema and aggravate COPD (7-10). Smoking, leading to emphysema, constitutes an additional negative factor that contributes to the decrease in lung diffusing capacity (11, 12). Importantly, the etiology of this disease is unknown and there is no treatment capable of reversing the pathology of COPD. In the advanced forms, the only hope is lung transplantation.

Another severe disease is idiopathic pulmonary fibrosis (IPF); it occurs mostly in patients 60 years of age and older and carries a high mortality rate (13-15). Genetic factors, environmental insults and viral infections have been claimed to contribute to the onset and evolution of IPF (14). Mutations of telomerase and telomere shortening have been found with IPF (16-24). As for COPD, currently there is no well-established treatment for IPF and none of the available therapies prolongs survival in this patient population (25). Similarly, secondary progressive pulmonary fibrosis (PPF) has devastating clinical consequences (26-29). It is incontrovertible that COPD and IPF/PPF require the implementation of new strategies to define their pathophysiology and develop innovative forms of treatment.

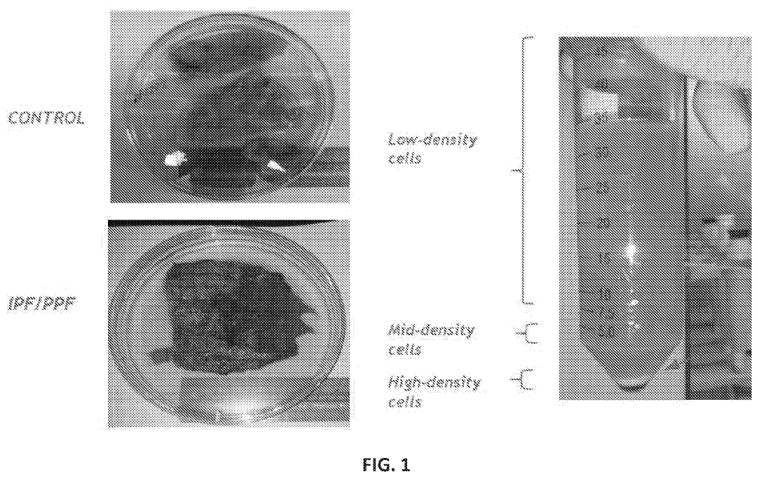

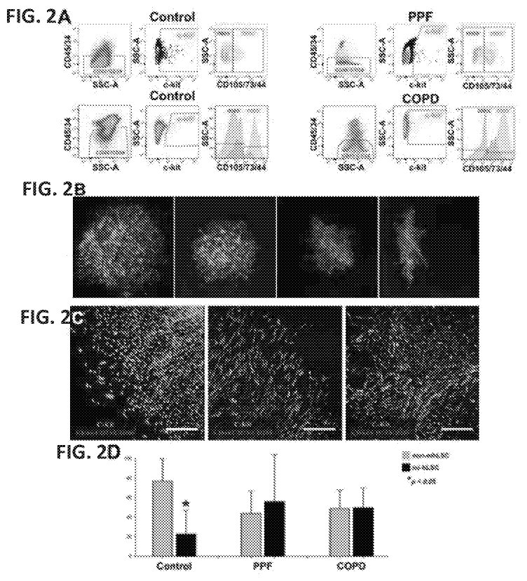

Recently, control lungs declined for transplantation (n=13), and IPF/PPF (n=8) and COPD (n=7) explanted lungs were studied (FIG. 1). The inventors have found that a pool of c-kit-positive human lung stem cells (hLSCs) is composed of one cell class that is negative for the mesenchymal epitopes CD44/CD73/CD105, i.e., non-mesenchymal hLSCs (non-mhLSCs), and another cell class that expresses these epitopes (FIG. 2A) and differentiates into adipocytes, chondrocytes and osteocytes, i.e., mesenchymal-like hLSCs (ml-hLSCs). Both cell types possess the properties of tissue specific adult stem cells, i.e., self-renewal and clonogenicity (FIGS. 2B and 2C). The majority of clones derived from control and IPF/PPF non-mhLSCs displayed features of stem cell-formed colonies; they had a compact round shape (FIG. 2B; two left panels). Non-circular irregularly shaped clones with refractive edges were occasionally found with control non-mhLSCs but reached a value of 29% with IPF/PPF non-mhLSCs (FIG. 2B; two right panels). Conversely, control and IPF/PPF mhLSCs formed only non-circular clones. Importantly, the circular clones were composed of undifferentiated cells intensely positive for c-kit, high nucleus-to-cytoplasm ratio and negative for the mesenchymal epitopes CD44/CD73/CD105 (FIG. 2C; left panel). The non-circular clones, however, were characterized by cells weakly labeled for c-kit, low nucleus-to-cytoplasm ratio and positive for CD44/CD73/CD105 (FIG. 2C; central and right panels). The proportion of non-mhLSCs (77%) and ml-hLSCs (23%) in control lungs changes significantly with IPF/PPF and COPD where ml-hLSCs and non-mhLSCs are nearly 50% each (FIG. 2D).

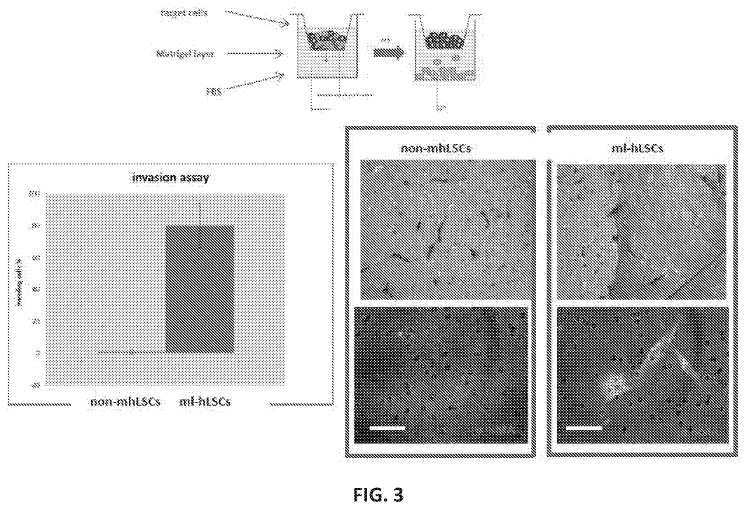

Of relevance, clonal non-mhLSCs differentiate in alveolar epithelial cells and capillary endothelial cells (not shown), while clonal ml-hLSCs do not acquire the epithelial and vascular cell lineages. ml-hLSCs from IPF/PPF lungs generate a large number of fibroblasts/myofibroblasts and invade the matrigel at high rate, acquiring the myofibroblast phenotype (FIG. 3). These data indicate that with IPF/PPF, ml-hLSCs possess characteristics which make them a candidate of lung pathology. With COPD, the increase in ml-hLSCs and the decrease in non-mhLSC attenuate the ability of the COPD lung to form gas exchange units and this may lead to enlargement of alveoli, destruction of the alveolar wall and respiratory failure.

Importantly, a subset of functional non-mhLSCs is present in the IPF/PPF and COPD lung and, as shown here, these cells can be harvested and propagated in vitro. In the future, it should be possible to implement autologous cell therapy in an effort to reverse the devastating consequences of IPF/PPF and COPD.

### REFERENCES

1. Salazar L M, Herrera A M. Fibrotic response of tissue remodeling in COPD. Lung. 2011; 189:101-109.
2. Burgel P R, Bourdin A, Chanez P, Chabot F, Chaouat A, Chinet T, de Blic J, Devillier P, Deschildre A, Didier A, Garcia G, Jebrak G, Laurent F, Morel H, Perez T, Pilette C, Roche N, Tillie-Leblond I, Verbanck S, Dusser D. Update on the roles of distal airways in COPD. Eur Respir Rev. 2011; 20:7-22
3. Hinata N, Martin J G. Mechanisms of airway remodeling. Chest. 2013; 144:1026-1032.
4. Sohal S S, Ward C, Danial W, Wood-Baker R, Walters E H. Recent advances in understanding inflammation and remodeling in the airways in chronic obstructive pulmonary disease. Expert Rev Respir Med. 2013; 7:275-288.
5. Bagdonas E, Raudoniute J, Bruzauskaite I, Aldonyte R. Novel aspects of pathogenesis and rcgcncration mechanisms in COPD. Int J Citron Obstruct Pulmon Dis. 2015; 10:995-1013.
6. Barnes P J. Inflammatory mechanisms in patients with COPD. J Allergy Clin Immunol. 2016; 138:16-27.
7. Tsuji T, Aoshiba K, Nagai A. Alveolar cell senescence in patients with pulmonary emphysema. Am J Respir Crit Care Med. 2006; 174:886-893.
8. Tsuji T, Aoshiba K, Nagai A. Alveolar cell senescence exacerbates pulmonary inflammation in patients with chronic obstructive pulmonary disease. Respiration. 2010; 80:59-70.
9. Alder J K, Guo N, Kembou F, Parry E M, Anderson C J, Gorgy A I, Walsh M F, Sussan T, Biswal S, Mitzner W, Tuder R M, Armanios M. Telomere length is a determinant of emphysema susceptibility. Am J Respir Crit Care Med. 2011.
10. Amsellem V, Gary-Bobo G, Marcos E, Maitre B, Chaar V, Validire P, Stern J B, Noureddine H, Sapin E, Rideau D, Hue S, Le Corvoisier P, Le Gouvello S, Dubois-Rande J L, Boczkowski J, Adnot S, Telomere dysfunction causes sustained inflammation in chronic obstructive pulmonary disease. Am J Respir Crit Care Med. 2011; 184:1358-1366.
11. Viegi G, Sherrill D L, Carrozzi L, Di Pede F, Baldacci S, Pistelli F, Enright P. An 8-year follow-up of carbon monoxide diffusing capacity in a general population sample of northern Italy. Chest. 2001; 120:74-80.
12. Kapella M C, Larson J L, Covey M K, Alex C G. Functional performance in chronic obstructive pulmonary disease declines with time. Med Sci Sports Exerc. 2011; 43:218-224.
13. Mulugeta S, Nureki S, Beers M F. Lost after translation: insights from pulmonary surfactant for understanding the role of alveolar epithelial dysfunction and cellular quality control in fibrotic lung disease. Am J Physiol Lung Cell Mol Physiol. 2015; 309:L507-525.
14. Elmufdi F, Henke C A, Perlman D M, Tomic R, Kim H J. Novel mechanisms and treatment of idiopathic pulmonary fibrosis. Discov Med. 2015; 20:145-153.
15. Sgalla G, Bill A, Richeldi L. Idiopathic pulmonary fibrosis: Diagnosis, epidemiology and natural history. Respirology. 2016; 21:427-437.
16. Calado R T, Young N S. Telomere diseases. N Engl J Med. 2009; 361:2353-2365.
17. Mushiroda T, Wattanapokayakit S, Takahashi A, Nukiwa T, Kudoh S, Ogura T, Taniguchi H, Kubo M, Kamatani N, Nakamura Y; Pirfenidone Clinical Study Group. A genome-wide association study identifies an association of a common variant in TERT with susceptibility to idiopathic pulmonary fibrosis. J Med Genet. 2008; 45:654-656.
18. Cronkhite J T, Xing C, Raghu G. Chin K M, Tones F, Rosenblatt R L, Garcia C K. Telomere shortening in familial and sporadic pulmonary fibrosis. Am J Respir Crit Care Med. 2008; 178:729-737.
19. Bone R, Crestani B, Bichat H. Prevalence of telomere shortening in familial and sporadic pulmonary fibrosis is increased in men. Am J Respir Crit Care Med. 2009; 179:1073.
20. Waisberg D R, Baibas-Filho J V, Parra E R, Fernezlian S, de Carvalho C R, Kairalla R A, Capelozzi V L. Abnormal expression of telomerase/apoptosis limits type I I alveolar epithelial cell replication in the early remodeling of usual interstitial pneumonia/idiopathic pulmonary fibrosis. Hum Pathol. 2010; 41:385-391.
21. Armanios M. Telomerase and idiopathic pulmonary fibrosis. Mutat Res. 2012; 730:52-58.
22. Alder J K, Cogan J D, Brown A F, Anderson C J, Lawson W E, Lansdorp P M, Phillips J A 3rd, Loyd J E, Chen J J, Armanios M. Ancestral mutation in telomerase causes defects in repeat addition processivity and manifests as familial pulmonary fibrosis. PLoS Genet. 2011; 7:e1001352.
23. El-Chemaly S, Ziegler S G, Calado R T, Wilson K A, Wu H P, Haughey M, Peterson N R, Young N S, Gahl W A, Moss J, Gochuico B R. Natural history of pulmonary fibrosis in two subjects with the same telomerase mutation. Chest 2011; 139:1203-1209.
24. Diaz de Leon A, Cronkhite J T, Yilmaz C, Brewington C, Wang R, Xing C, Hsia C C, Garcia C K. Subclinical lung disease, macrocytosis, and premature graying in kindreds with telomerase (TERT) mutations. Chest. 2011; 140:753-763.
25. Canestaro W J, Forrester S H, Raghu G, Ho L, Devine B E. Drug treatment of idiopathic pulmonary fibrosis: systematic review and network meta-analysis. Chest. 2016; 149:756-766.
26. Veeraraghavan S, Nicholson A G, Wells A U. Lung fibrosis: new classifications and therapy. Curr Opin Rheumatol. 2001; 13:500-504.
27. de Lauretis A, Veeraraghavan S, Renzoni E. Review series: Aspects of interstitial lung disease: connective tissue disease-associated interstitial lung disease: how does it differ from IPF? How should the clinical approach differ? Chron Respir Dis. 2011; 8:53-82.
28. Katzenstein A L. Smoking-related interstitial fibrosis (SRIF), pathogenesis and treatment of usual interstitial pneumonia (UIP), and transbronchial biopsy in UIP. Mod Pathol. 2012; 25 Suppl 1: S68-78.
29. Noble P W, Barkauskas C E, Jiang D. Pulmonary fibrosis: patterns and perpetrators. J Clin Invest. 2012; 122:2756-2762.

TABLE 1
Standard therapy for some lung diseases.

| Lung disease | Standard therapies |
|---|---|
| Chronic obstructive pulmonary diseases (COPD, | See below |
| which include diseases such as emphysema, | |
| chronic bronchitis, and asthma) | |
| Emphysema | Inhaled bronchodilators, inhaled glucocorticoids, |
| (either due to smoking, or alpha 1 anti-trypsin | oxygen therapy if severe disease. However none |
| deficiency) | of these therapies are curative or reverse the |
| | disease. Replacement with alpha-1 antiprotease |
| | if deficient. Ultimately patients with severe |
| | progressive disease may be considered for lung |
| | transplantation. |
| Chronic bronchitis | Inhaled bronchodilators, inhaled glucocorticoids, |
| | oxygen therapy if severe disease. Antibiotics |
| | intermittently. However none of these therapies |
| | are curative or reverse the disease. Ultimately |
| | patients with severe progressive disease may be |
| | considered for lung transplantation. |
| Asthma | Inhaled glucocorticoids, inhaled bronchodilators, |
| | leukotriene modifiers. |
| Pulmonary fibrosis | No therapy proven to be efficacious -- |
| | physicians will often try immunosuppressive |
| | agents or antioxidants. Supportive care including |
| | supplemental oxygen. Ultimately patients with |
| | progressive disease are considered for lung |
| | transplantation. |
| Interstitial pneumonias | Therapies include immunosuppressive agents, |
| Other interstitial lung diseases due to a variety of | quit smoking, removal from environmental |
| reasons including rheumatologic/immunologic | source. However, if the disease is progressive |
| diseases, smoking, exposure to environmental | lung transplant may need to be considered. |
| factors, or idiopathic. | |
| Lymphangioleiomyomatosis (LAM) | Hormonal manipulation, Sirolimus, lung |
| | transplantation when disease progressive. |
| Cystic fibrosis | Antibiotics, bronchodilators, agents to promote |
| | airway clearance of thick secretions, chest |
| | physiotherapy, glucocorticoids and supplemental |
| | oxygen if severe, and with time patients are |
| | often considered for lung transplantation. |
| Sarcoidosis | Immunosuppressive agents. If progressive and |
| | not responsive to therapy, consideration for lung |
| | transplantation. |
| Pulmonary hypertension | Oral vasodilators (only affective in a minority of |
| | patients), Prostanoid formulations (either inhaled |
| | or intravenous), endothelin receptor inhibitors, |
| | PDE5 inhibitors, combination therapies of the |
| | drug classes mentioned, supplemental oxygen, |
| | and anticoagulation - unfortunately patients |
| | progress and may be considered for lung |
| | transplantation. |
| Pulmonary veno-occlusive disease | Vasodilators, immunosuppressives, |
| | anticoagulants, and oxygen. Therapy shown to |
| | significantly prolong survival is lung |
| | transplantation. |
| Obliterative bronchiolitis (OB) - occurs due to | Immunosuppressive agents, patients may require |
| rejection after lung transplantation. Even though | repeat lung transplantation |
| transplant is definitive therapy for many | |
| progressive lung diseases, the 5-year survival is | |
| only 50%. | |

1. A pharmaceutical composition comprising:
an enriched population of isolated c-kit positive lung stem cells from a human lung tissue sample wherein the c-kit positive lung stem cells are negative for the CD44, CD73 and CD105 markers of the mesenchymal stromal cell lineage (non-mhLSCs); and
a pharmaceutically acceptable carrier.

2. The pharmaceutical composition of claim 1, wherein the lung tissue is from a subject of any age.

3. The pharmaceutical composition of claim 1, wherein the population of non-mhLSCs can differentiate into alveolar epithelial cells, capillary endothelial cells, or a combination thereof.

4. The pharmaceutical composition of claim 1, wherein the population of non-mhLSCs is self-renewing and clonogenic.

5. (canceled)

6. (canceled)

7. (canceled)

8. A method of preparing an isolated population of lung stem cells positive for c-kit and negative for the CD44, CD73 and CD105 markers of the mesenchymal stromal cell lineage (non-mhLSCs), wherein the non-mhLSCs are in a pool of c-kit-positive human lung stem cells (hLSCs) comprising non-mhLSCs and mesenchymal-like lung stem cells that are positive for c-kit and the CD44, CD73 and CD105 markers (ml-hLSCs), the method comprising:
a. obtaining human lung tissue from a subject;
b. selecting non-mhLSCs from the pool of hLSCs from the human lung tissue; and
c. proliferating said cells in a culture medium.

9. (canceled)

10. (canceled)

11. (canceled)

12. The method of claim 8, wherein the human lung tissue is an adult or a non-adult lung tissue.

13. The method of claim 8, wherein the human lung tissue is cryopreserved prior to selecting or extracting the non-mhLSCs.

14. The method of claim 8, wherein the selecting or extracting of non-mhLSCs is performed using an antibody against c-kit.

15. The method of claim 8, further comprising negative selection for the CD44, CD73 and CD105 markers of the mesenchymal stromal cell lineage.

16-46. (canceled)

47. A method for treating or preventing a lung disease or disorder in a subject in need thereof, comprising:
a. obtaining a human lung tissue from the subject in need thereof or from a different subject;
b. extracting a population of stern cells positive for c-kit and negative for the CD44, CD73 and CD105 markers of the mesenchymal stromal cell lineage (non-mhLSCs) from said lung tissue;
c. expanding said population of non-mhLSCs; arid
d. administering said expanded population of non-mhLSCs to the subject in need thereof.

48. The method of claim 47, further comprising selecting a subject who is suffering from a lung disease or disorder prior to administering the population of non-mhLSCs.

49. The method of claim 47, wherein the lung disease or disorder is one or more of chronic obstructive pulmonary disease (COPD), idiopathic pulmonary fibrosis (IPF), or progressive pulmonary fibrosis (PPF).
